(19) United States
(12) Patent Application Publication
(10) Pub. No.: US 2003/0119806 A1
(43) Pub. Date: Jun. 26, 2003

(54) Azetidine derivatives, processes for their preparation and their use as pesticides

(76) Inventors: Stephen Lindell, Kelkheim-Fischbach (DE); Joachim Dickhaut, Heidelberg (DE); Harald Jakobi, Frankfurt (DE); Jorg Tiebes, Frankfurt (DE); Waltraud Hempel, Liederbach (DE); Daniela Jans, Bad Homburg v. d. H. (DE); Jutta Maria Waibel, Frankfurt (DE)

Correspondence Address: Eric H. Weisblatt, BURNS, DOANE, SWECKER & MATHIS, L.L.P., P.O. Box 1404, Alexandria, VA, 22313-1404, US

(21) Appl. No.: 10/237,604

(22) Filed: Sep. 10, 2002

(30) Foreign Application Priority Data
Sep. 12, 2001 (DE) 10144872.4

(51) Int. Cl. A61K 31/397; A61K 31/4439; C 07D 4 1/02; C07D 205/00

(52) U.S. Cl. 514/210.01; 514/210.2; 548/950; 546/268.1

(57) ABSTRACT

Compounds of the formula (I)

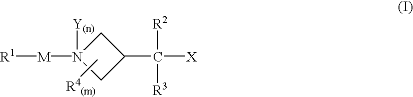

in which the symbols and indices are as defined below:

R¹ is aryl or heteroaryl, which are unsubstituted or mono- or polysubstituted by identical or different substituents;

R² and R³ are aryl or heteroaryl, which are unsubstituted or mono- or polysubstituted by identical or different substituents, it also being possible for the two groups to be bridged by a joint substituent;

M is unsubstituted or substituted (CH₂)ₗ, where l=1, 2 or 3, is CO or —HN—C(O);

X is H, OH, halogen, OR⁴ or CN;

Y is (O), H, OH, OR⁴, R⁴; in the case of the four lastmentioned groups—in which the nitrogen carries a positive charge—in combination with a corresponding anion;

R⁴ are identical or different (C₁-C₄)-alkyl, (C₁-C₄)-alkanoyl or (C₁-C₄)-haloalkyl groups;

m is 0, 1, 2, 3 or 4;

n is 0 or 1;

are useful as pesticides.

The invention relates to azetidine derivatives, to processes for their preparation, to compositions comprising them and to their use for controlling animal pests, in particular arthropods, such as insects and Acarina, and helminths.

Owing to their biological activity, certain N-benzylpiperidine derivatives are suitable for controlling animal pests (see, for example, WO-A-95/23507, WO-A-96/36228, WO-A-97/26252, WO-A-98/00015, WO-A-00/31034 and WO-A-01/17964).

However, owing to the multifarious requirements that modern pesticides have to meet, for example with respect to efficacy, persistency, activity spectrum, use spectrum, toxicity, combination with other active compounds, combination with formulating agents or synthesis, and owing to the possible occurrence of resistance, the development of such substances can never be considered to be concluded, and there is a constant great need for novel compounds which, at least in some aspects, offer advantages compared to the known compounds.

It was an object of the present invention to provide compounds which, under various aspects, widen the spectrum of pesticides.

This object and other objects which have not been explicitly mentioned, which can be derived or deduced from the context discussed here, are achieved by the azetidine derivatives of the formula (I).

Azetidine derivatives for treating cardiac arrhythmias are known, for example, from DE-A 28 18 672. That such compounds may be suitable for use as pesticides cannot be derived from this application.

Accordingly, the present invention provides azetidine derivatives of the formula (I)

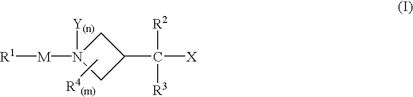

in which the symbols and indices are as defined below:

R¹ is aryl or heteroaryl, which are unsubstituted or mono- or polysubstituted by identical or different substituents;

R² and R³ are aryl or heteroaryl, which are unsubstituted or mono- or polysubstituted by identical or different substituents, it also being possible for the two groups to be bridged by a joint substituent;

M is unsubstituted or substituted (CH₂)ₗ, where l=1, 2 or 3, is CO or —HN—C(O);

X is H, OH, halogen, OR⁴ or CN;

Y is O, H, OH, OR⁴, R⁴; in the case of the four lastmentioned groups—in which the nitrogen carries a positive charge—in combination with a corresponding anion;

R⁴ are identical or different (C₁-C₄)-alkyl, (C₁-C₄)-alkanoyl or (C₁-C₄)-haloalkyl groups;

m is 0, 1, 2, 3 or 4;

n is 0 or 1;

except for compounds in which

Y is CN,

R¹ is unsubstituted phenyl,

R² is unsubstituted phenyl,

R³ is unsubstituted phenyl or pyridyl and,

n is 0.

The symbols and indices in the formula (I) preferably have the following meanings:

R¹, R², R³ are preferably independently of one another carbocyclic aromatic radicals having 6 to 14, preferably 6 to 12, particularly preferably 6 to 10, carbon atoms, or aryl radicals having 6 to 14, preferably 6 to 12, particularly preferably 6 to 10, ring members, where at least one CH group is replaced by N and/or two adjacent CH groups are replaced by S, NH or O and where the radicals mentioned are unsubstituted or mono- or polysubstituted by identical or different substituents.

R¹, R², R³ are particularly preferably phenyl, naphthyl, thiophene, furan, pyrrole, thiazole, oxazole, imidazole, isothiazole, isoxazole, pyrazole, 1,3,4-oxadiazole, 1,3,4-thiadiazole, 1,3,4-triazole, 1,2,4-oxadiazole, 1,2,4-thiadiazole, 1,2,4-triazole, 1,2,3-triazole, 1,2,3,4-tetrazole, benzo[b]thiophene, benzo[b]furan, indole, benzo[c]thiophene, benzo[c]furan, isoindole, benzoxazole, benzothiazole, benzimidazole, benzisoxazole, benzisothiazole, benzopyrazole, benzothiadiazole, benzotriazole, dibenzofuran, dibenzothiophene, carbazole, pyridine, pyrazine, pyrimidine, pyridazine, 1,3,5-triazine, 1,2,4-triazine, 1,2,4,5-triazine, quinoline, isoquinoline, quinoxaline, quinazoline, cinnoline, 1,8-naphthyridine, 1,5-naphthyridine, 1,6-naphthyridine, 1,7-naphthyridine, phthalazine, pyridopyrimidine, purine, pteridine or 4H-quinolizine.

R¹ is very particularly preferably phenyl, pyridyl, in particular 2-pyridyl, thienyl, in particular 2-thienyl, furanyl, in particular 2-furanyl, pyrimidyl, in particular 2- or 4-pyrimidyl; with particular preference phenyl.

R², R³ are identical or different, preferably identical, and are very particularly preferably phenyl, pyridyl, in particular 2-pyridyl, thienyl, in particular 2-thienyl, or furanyl, in particular 2-furanyl; with particular preference phenyl.

M is preferably —CH₂—, —CH₂—CH₂—, —CH₂—CH₂—CH₂—, —CH(CH₃)—, —NH—CO—, —CO— or

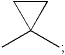

;

particularly preferably —CH₂—, —CH₂—CH₂—, —CH₂—CH₂—CH₂—; very particularly preferably —CH₂—.

X is preferably H, Cl, F, OH, CN, particularly preferably F, OH, H, very particularly preferably OH.

Y is preferably O, OH or H.

R⁴ is preferably Me.

m is preferably O.

Identical or different substituents in which the radicals R¹, R² and R³ may be mono- or polysubstituted are preferably radicals R⁵.

R⁵ are identical or different

a) halogen, CN, NO₂, SF₅;

b) straight-chain or branched alkyl groups having 1 to 12 carbon atoms, where one or more (CH₂) groups may be replaced by —O—, —S(O)—_{0,1,2}, —NH—, —NR⁶—, —CO—, —CS—, —CH═CH—, —C≡C—, unsubstituted or substituted aryidiyl, unsubstituted or substituted heterocyclyldiyl, unsubstituted or substituted (C₃-C₈)-cycloalkanediyl or unsubstituted or substituted (C₃-C₈)-cycloalkenediyl, with the proviso that chalcogen may not be adjacent, where two radicals R⁵ together with the atoms of the aromatic ring system may form a 3- to 8-membered ring system and where individual hydrogen atoms may be replaced by halogen, CN, NO₂ and/or SF₅;

with the proviso that the radical(s) R⁵ together do not comprise more than 3 ring systems having five or more members.

R⁶ is (C₁-C₄)-alkyl, unsubstituted or substituted phenyl or unsubstituted or substituted benzyl.

As substituents on the radicals R¹, R², R³, the radicals R⁵ particularly preferably have the following meanings:

R⁵ are identical or different D-R⁷, or two radicals R⁵ together with the atoms to which they are attached form a three- to eight-membered saturated or unsaturated ring system which may also comprise further heteroatoms, preferably O, N, S, SO and/or SO₂, and which is unsubstituted or substituted by one or more radicals R⁷;

D is a direct bond or (C₁-C₆)-alkanediyl which is unsubstituted or substituted by one or more halogen atoms;

R⁷ are identical or different R⁸, R⁹, —C(W)R⁸, —C(═NOR⁸)R⁸, —C(═NR⁸)R⁸, —C(═NNR⁸₂)R⁸, —C(═W)OR⁸, —C(═W)NR⁸₂, —OC(═W)R⁸, —OC(═W)OR⁸, —NR⁸C(═W)R⁸, —N[C(═W)R⁸]₂, —NR⁸C(═W)OR⁸, —C(═W)NR⁸—NR⁸₂, —C(═W)NR⁸—NR⁸[C(═W)R⁸], —NR⁸—C(═W)NR⁸₂, —NR⁸—NR⁸C(═W)R⁸, —NR⁸—N[C(═W)R⁸]₂, —N[(C═W)R⁸]—NR⁸₂, —NR⁸—N[(C═W)WR⁸], —NR⁸[(C═W)NR⁸₂], —NR⁸(C═NR⁸)R⁸, —NR⁸(C═NR⁸)NR⁸₂, —O—NR⁸₂, —O—NR⁸(C═W)R⁸, —SO₂NR⁸₂, —NR⁸SO₂R⁸, —SO₂OR⁸, —OSO₂R⁸, —OR⁸, —NR⁸₂, —SR⁸, —SiR⁸₃, —PR⁸₂, —P(═W)R⁸₂, —SOR⁸, —SO₂R⁸, —PW₂R⁸₂, —PW₃R⁸₂ group;

W is O or S;

R⁸ are identical or different H, (C₁-C₆)-alkyl, (C₂-C₆)-alkenyl, (C₂-C₆)-alkynyl, (C₃-C₈)-cycloalkyl, (C₄-C₈)-cycloalkenyl, (C₃-C₈)-cycloalkyl-(C₁-C₄)-alkyl, (C₄-C₈)-cycloalkenyl-(C₁-C₄)-alkyl, (C₃-C₈)-cycloalkyl-(C₂-C₄)-alkenyl, (C₄-C₈)-cycloalkenyl-(C₂-C₄)-alkenyl, (C₁-C₆)-alkyl-(C₃-C₈)-cycloalkyl, (C₂-C₆)-alkenyl-(C₃-C₈)-cycloalkyl, (C₂-C₆)-alkynyl-(C₃-C₈)-cycloalkyl, (C₁-C₆)-alkyl-(C₄-C₈)-cycloalkenyl, (C₂-C₆)-alkenyl-(C₄-C₈)-cycloalkenyl, aryl or heterocyclyl groups, where the radicals mentioned are unsubstituted or substituted by one or more radicals R⁹ and two radicals R together may form a ring system;

R⁹ are identical or different halogen, cyano, nitro, hydroxyl, thio, amino, SF₅, formyl, (C₁-C₆)-alkanoyl, (C₁-C₆)-alkoxy, (C₃-C₆)-alkenyloxy, (C₃-C₆)-alkynyloxy, (C₁-C₆)-haloalkyloxy, (C₃-C₆)-haloalkenyloxy, (C₃-C₆)-haloalkynyloxy, (C₃-C₈)-cycloalkoxy, (C₄-C₈)-cycloalkenyloxy, (C₃-C₈)-halocycloalkoxy, (C₄-C₈)-halocycloalkenyloxy, (C₃-C₈)-cycloalkyl-(C₁-C₄)-alkoxy, (C₄-C₈)-cycloalkenyl-(C₁-C₄)-alkoxy, (C₃-C₈)-cycloalkyl-(C₂-C₄)-alkenyloxy, (C₄-C₈)-cycloalkenyl-(C₁-C₄)-alkenyloxy, (C₁-C₆)-alkyl-(C₃-C₈)-cycloalkoxy, (C₂-C₆)-alkenyl-(C₃-C₈)-cycloalkoxy, (C₂-C₆)-alkynyl-(C₃-C₈)-cycloalkoxy, (C₁-C₆)-alkyl-(C₄-C₈)-cycloalkenyloxy, (C₂-C₆)-alkenyl-(C₄-C₈)-cycloalkenyloxy, (C₁-C₄)-alkoxy-(C₁-C₆)-alkoxy, (C₁-C₄)-alkoxy-(C₃-C₆)-alkenyloxy, carbamoyl, (C₁-C₆)-mono- or -dialkylcarbamoyl, (C₁-C₆)-mono- or -dihaloalkylcarbamoyl, (C₃-C₈)-mono- or -dicycloalkylcarbamoyl, (C₁-C₆)-alkoxycarbonyl, (C₃-C₈)-cycloalkoxycarbonyl, (C₁-C₆)-alkanoyloxy, (C₃-C₈)-cycloalkanoyloxy, (C₁-C₆)-haloalkoxycarbonyl, (C₁-C₆)-haloalkanoyloxy, (C₁-C₆)-alkaneamido, (C₁-C₆)-haloalkaneamido, C(O)NH—(C₁-C₆)-alkyl, C(O)NH—(C₁-C₆)-haloalkyl, C(O)N-[(C₁-C₆)-alkyl]₂, C(O)N-[(C₁-C₆)-haloalkyl]₂, (C₂-C₆)-alkeneamido, (C₃-C₈)-cycloalkaneamido, (C₃-C₈)-cycloalkyl-(C₁-C₄)-alkaneamido, (C₁-C₆)-alkylthio, (C₃-C₆)-alkenylthio, (C₃-C₆)-alkynylthio, (C₁-C₆)-haloalkylthio, (C₃-C₆)-haloalkenylthio, (C₃-C₆)-haloalkynylthio, (C₃-C₈)-cycloalkylthio, (C₄-C₈)-cycloalkenylthio,

(C₃-C₈)-halocycloalkylthio, (C₄-C₈)-halocycloalkenylthio, (C₃-C₈)-cycloalkyl-(C₁-C₄)-alkylthio, (C₄-C₈)-cycloalkenyl-(C₁-C₄)-alkylthio, (C₃-C₈)-cycloalkyl-(C₃-C₄)-alkenylthio, (C₄-C₈)-cycloalkenyl-(C₃-C₄)-alkenylthio, (C₁-C₆)-alkyl-(C₃-C₈)-cycloalkylthio, (C₂-C₆)-alkenyl-(C₃-C₈)-cycloalkylthio, (C₂-C₆)-alkynyl-(C₃-C₈)-cycloalkylthio, (C₁-C₆)-alkyl-(C₄-C₈)-cycloalkenylthio, (C₂-C₆)-alkenyl-(C₄-C₈)-cycloalkenylthio, (C₁-C₆)-alkylsulfinyl, (C₃-C₆)-alkenylsulfinyl, (C₃-C₆)-alkynylsulfinyl, (C₁-C₆)-haloalkylsulfinyl, (C₃-C₆)-haloalkenylsulfinyl, (C₃-C₆)-haloalkynylsulfinyl, (C₃-C₈)-cycloalkylsulfinyl, (C₄-C₈)-cycloalkenylsulfinyl, (C₃-C₈)-halocycloalkylsulfinyl, (C₄-C₈)-halocycloalkenylsulfinyl, (C₃-C₈)-cycloalkyl-(C₁-C₄)-alkylsulfinyl, (C₄-C₈)-cycloalkenyl-(C₁-C₄)-alkylsulfinyl, (C₃-C₈)-cycloalkyl-(C₃-C₄)-alkenylsulfinyl, (C₄-C₈)-cycloalkenyl-(C₃-C₄)-alkenylsulfinyl, (C₁-C₆)-alkyl-(C₃-C₈)-cycloalkylsulfinyl, (C₂-C₆)-alkenyl-(C₃-C₈)-cycloalkylsulfinyl, (C₂-C₆)-alkynyl-(C₃-C₈)-cycloalkylsulfinyl, (C₁-C₆)-alkyl-(C₄-C₈)-cycloalkenylsulfinyl, (C₂-C₆)-alkenyl-(C₄-C₈)-cycloalkenylsulfinyl, (C₁-C₆)-alkylsulfonyl, (C₃-C₆)-alkenylsulfonyl, (C₃-C₆)-alkynylsulfonyl, (C₁-C₆)-haloalkylsulfonyl, (C₃-C₆)-haloalkenylsulfonyl, (C₃-C₆)-haloalkynylsulfonyl, (C₃-C₈)-cycloalkylsulfonyl, (C₄-C₈)-cycloalkenylsulfonyl, (C₃-C₈)-halocycloalkylsulfonyl, (C₄-C₈)-halocycloalkenylsulfonyl, (C₃-C₈)-cycloalkyl-(C₁-C₄)-alkylsulfonyl, (C₄-C₈)-cycloalkenyl-(C₁-C₄)-alkylsulfonyl, (C₃-C₈)-cycloalkyl-(C₃-C₄)-alkenylsulfonyl, (C₄-C₈)-cycloalkenyl-(C₃-C₄)-alkenylsulfonyl, (C₁-C₆)-alkyl-(C₃-C₈)-cycloalkylsulfonyl, (C₂-C₆)-alkenyl-(C₃-C₈)-cycloalkylsulfonyl, (C₂-C₆)-alkynyl-(C₃-C₈)-cycloalkylsulfonyl, (C₁-C₆)-alkyl-(C₄-C₈)-cycloalkenylsulfonyl, (C₂-C₆)-alkenyl-(C₄-C₈)-cycloalkenylsulfonyl, (C₁-C₆)-dialkylamino, (C₁-C₆)-alkylamino, (C₃-C₆)-alkenylamino, (C₃-C₆)-alkynylamino, (C₁-C₆)-haloalkylamino, (C₃-C₆)-haloalkenylamino, (C₃-C₆)-haloalkynylamino, (C₃-C₈)-cycloalkylamino, (C₄-C₈)-cycloalkenylamino, (C₃-C₈)-halocycloalkylamino, (C₄-C₈)-halocycloalkenylamino, (C₃-C₈)-cycloalkyl-(C₁-C₄)-alkylamino, (C₄-C₈)-cycloalkenyl-(C₁-C₄)-alkylamino, (C₃-C₈)-cycloalkyl-(C₃-C₄)-alkenylamino, (C₄-C₈)-cycloalkenyl-(C₃-C₄)-alkenylamino, (C₁-C₆)-alkyl-(C₃-C₈)-cycloalkylamino, (C₂-C₆)-alkenyl-(C₃-C₈)-cycloalkylamino, (C₂-C₆)-alkynyl-(C₃-C₈)-cycloalkylamino, (C₁-C₆)-alkyl-(C₄-C₈)-cycloalkenylamino, (C₂-C₆)-alkenyl-(C₄-C₈)-cycloalkenylamino, (C₁-C₆)-trialkylsilyl, aryl, aryloxy, arylthio, arylamino, aryl-(C₁-C₄)-alkoxy, aryl-(C₃-C₄)-alkenyloxy, aryl-(C₁-C₄)-alkylthio, aryl-(C₂-C₄)-alkenylthio, aryl-(C₁-C₄)-alkylamino, aryl-(C₃-C₄)-alkenylamino, aryl-(C₁-C₆)-dialkylsilyl, diaryl-(C₁-C₆)-alkylsilyl, triarylsilyl or 5- or 6-membered heterocyclyl groups, where the cyclic radicals are unsubstituted or substituted by one or more radicals selected from the group consisting of halogen, cyano, nitro, amino, hydroxy, thio, (C₁-C₄)-alkyl, (C₁-C₄)-haloalkyl, (C₃-C₈)-cycloalkyl, (C₁-C₄)-alkoxy, (C₁-C₄)-haloalkoxy, (C₁-C₄)-alkylthio, (C₁-C₄)-haloalkylthio, (C₁-C₄)-alkylamino, (C₁-C₄)-haloalkylamino, formyl and (C₂-C₄)-alkanoyl.

As substituent on the radical R¹, R⁵ very particularly preferably has the following meaning: halogen, (C₁-C₄)-alkoxy, (C₁-C₄)-haloalkoxy, (C₃-C₆)-cyclopropyl-(C₁-C₄)-alkoxy, (C₃-C₆)-alkenyloxy, (C₁-C₄)-alkoxycarbonyl, —NHCO₂—(C₁-C₄)-alkyl, —NHCO₂—(C₁-C₄)-haloalkyl, —N═C((C₁-C₄)-alkyl)-COO(C₁-C₄)-alkyl, —N((C₁-C₄)-alkyl)-COO—(C₁-C₄)-haloalkyl, OC(O)NH—(C₁-C₄)-alkyl, —O—C(O)—N((C₁-C₄)-alkyl)₂, CH═NO—(C₁-C₄)-alkyl, —CH═N—(C₁-C₄)-alkyl, CH═N-phenyl, CH═N-heterocyclyl, aryloxy,

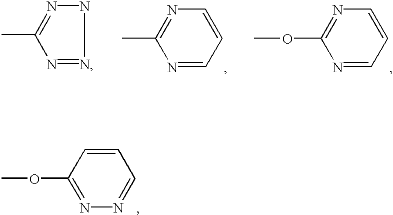

where the 5 lastmentioned groups are unsubstituted or mono- or—if possible—polysubstituted by identical or different radicals selected from the group consisting of halogen, (C₁-C₄)-alkyl, (C₁-C₄)-haloalkyl, (C₁-C₄)-alkoxy and (C₁-C₄)-haloalkoxy, or two radicals R⁵ together are —O—(CH₂)_{1,2}—O—.

Substituents on R¹ which are especially preferred are OEt, OⁿPr, OⁿBu, OⁱBu, O^{t}Bu, OCF₃, OCH₂CF₃, halogen,

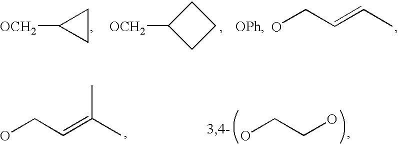

CO₂Et, NHCO₂Me, NHCO₂Et, NHCO₂ⁱPr, NHCO₂ⁿBu, NHCO₂CH₂CF₃, NⁱPrCO₂Me, OCO₂NMe₂, OCO₂NHMe, OCO₂NHEt, CH═NOEt, CH═NOⁿPr, CH═NMe, CH═NEt, CH═NⁱPr,

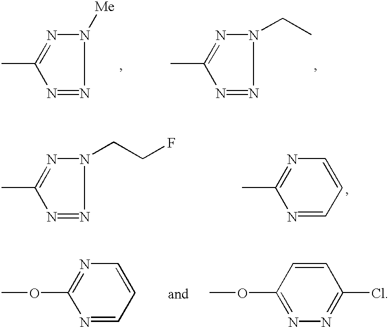

As substituents on groups R² and R³, the radicals R⁵, which can be identical or different, very particularly preferably have the following meanings: halogen, SF₅, (C₁-C₄)-haloalkyl, (C₁-C₄)-haloalkoxy, in particular in the 3- or 4-position of a phenyl group.

Especially preferred are CF₃, OCF₃, F, Cl, Br, I, SF₅, OCHF₂, OCH₂CF₃, in particular in the 3- or 4-position of a phenyl group; amongst these, in turn, preference is given to CF₃ and OCF₃, preferably in the 4-position of a phenyl group.

Preferred groups of compounds of the formula (I) are those of the formulae (Ia)-(Ic)

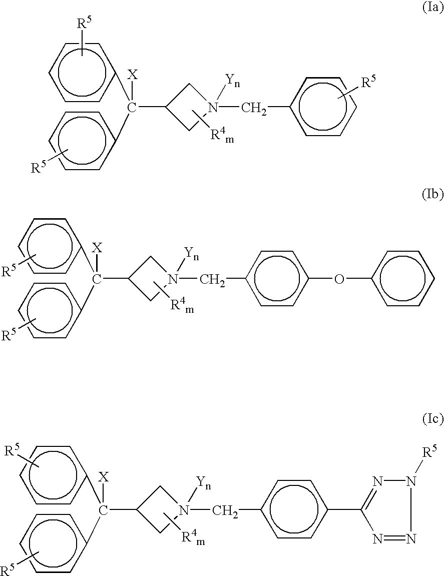

In the above formula, “halogen” is to be understood as meaning a fluorine, chlorine, bromine or iodine atom;

the term “(C₁-C₄)-alkyl” is to be understood as meaning an unbranched or branched hydrocarbon radical having 1 to 4 carbon atoms, such as, for example the methyl, ethyl, propyl, isopropyl, 1-butyl, 2-butyl, 2-methylpropyl or tert-butyl radical;

the term “(C₁-C₈)-alkyl” is to be understood as meaning the abovementioned alkyl radicals and also, for example, the pentyl, 2-methylbutyl, 1,1-dimethylpropyl, hexyl, heptyl, octyl or the 1,1,3,3-tetramethylbutyl radical;

alkanediyl is to be understood as meaning an unbranched or branched alkanediyl radical having 1 to 8 carbon atoms, such as, for example, methylene, ethane-1,2-diyl, propane-1,2-diyl, propane-1,3-diyl, butane-1,4-diyl, butane-1,3-diyl, butane-1,2-diyl, 2-methylpropane-1,2-diyl, 2-methylpropane-1,3-diyl;

the term “(C₁-C₄)-haloalkyl” is to be understood as meaning an alkyl group mentioned under the term “(C₁-C₄)-alkyl” in which one or more hydrogen atoms are replaced by the halogen atoms mentioned above, preferably by chlorine or fluorine, such as, for example, the trifluoromethyl group, the 1-fluoroethyl group, the 2,2,2-trifluoroethyl group, the chloromethyl group, the fluoromethyl group, the difluoromethyl group or the 1,1,2,2-tetrafluoroethyl group;

the term “(C₃-C₈)-cycloalkyl” is to be understood as meaning, for example, the cyclopropyl, cyclobutyl or cyclopentyl group; and also the cyclohexyl, cycloheptyl or cyclooctyl radical;

the term “(C₃-C₈)-halocycloalkyl” is to be understood as meaning one of the (C₃-C₅)-cycloalkyl radicals listed above in which one or more, in the case of fluorine optionally all, hydrogen atoms are replaced by halogen, preferably by fluorine or chlorine, such as, for example, the 2,2-difluoro- or 2,2-dichlorocyclopropane group or the fluorocyclopentane radical;

the term “(C₂-C₄)-alkenyl” is to be understood as meaning, for example, the vinyl, allyl, 2-methyl-2-propenyl or 2-butenyl group;

the term “(C₂-C₄)-haloalkenyl” is to be understood as meaning a (C₂-C₄)-alkenyl group in which some or, in the case of fluorine, also all hydrogen atoms are replaced by halogen, preferably by fluorine or chlorine;

the term “(C₂-C₄)-alkynyl” is to be understood as meaning, for example, the ethynyl, propargyl, 2-methyl-2-propynyl or 2-butynyl group;

the term “(C₂-C₈)-alkynyl” is to be understood as meaning, for example, the radicals mentioned above, and also, for example, the 1-pentynyl, 2-pentynyl, 3-pentynyl, 4-pentynyl or the 1-octynyl group;

the term “(C₂-C₄)-haloalkynyl” is to be understood as meaning a (C₂-C₄)-alkynyl group in which some of the hydrogen atoms, in the case of fluorine also all of the hydrogen atoms, are replaced by halogen atoms, preferably by fluorine or chlorine;

the term “(C₁-C₄)-hydroxyalkyl” is to be understood as meaning, for example, the hydroxymethyl, 1-hydroxyethyl, 2-hydroxyethyl, 1-hydroxy-1-methylethyl or the 1-hydroxypropyl group;

the term “(C₁-C₄)-alkanoyl” is to be understood as meaning, for example, the formyl, acetyl, propionyl, 2-methylpropionyl or the butyryl group;

the term “(C₁-C₈)-alkanoyl” is to be understood as meaning the radicals mentioned above and also, for example, the valeroyl, pivaloyl, hexanoyl, heptanoyl or octanoyl group;

the term “(C₁-C₁₂)-alkanoyl” is to be understood as meaning, for example, the radicals mentioned above and also, for example, the nonanoyl, decanoyl or the dodecanoyl group;

the term “(C₂-C₄)-haloalkanoyl” is to be understood as meaning a (C₁-C₄)-alkanoyl group in which some of the hydrogen atoms, in the case of fluorine also all, are replaced by halogen atoms, preferably by fluorine or chlorine;

the term “(C₂-C₁₂)-haloalkanoyl” is to be understood as meaning a (C₁-C₁₂)-alkanoyl group in which some of the hydrogen atoms, in the case of fluorine also all, are replaced by halogen atoms, preferably by fluorine or chlorine;

the term “(C₁-C₄)-alkanoyl-(C₁-C₄)-alkyl” is to be understood as meaning, for example, an acetylmethyl, propionylmethyl, 2-acetylethyl or a butyrylmethyl group;

the term “(C₁-C₆)-alkanoyloxy” is to be understood as meaning, for example, the methanoate, the ethanoate, the propanoate, the n-butanoate or the pentanoate or the hexanoate group;

the term “cyano-(C₁-C₄)-alkyl” is to be understood as meaning a cyanoalkyl group in which the hydrocarbon radical has the meanings given under the term “(C₁-C₄)-alkyl”;

the terms “(C₀-C₄)-nitroalkyl” and “(C₁-C₄)-thiocyanoalkyl” are to be understood as meaning one of the (C₁-C₄)-alkyl groups mentioned above which is substituted by a nitro and a thiocyano group, respectively;

the term “(C₁-C₄)-alkoxycarbonyl” is to be understood as meaning, for example, the methoxycarbonyl, ethoxycarbonyl, propoxycarbonyl, butoxycarbonyl or tert-butoxycarbonyl group;

the term “(C₁-C₈)-alkoxycarbonyl” is to be understood as meaning, for example, the radicals mentioned above, and also, for example, the pentyloxycarbonyl, hexyloxycarbonyl or the octyloxycarbonyl group;

the term “(C₁-C₁₂)-alkoxycarbonyl” is to be understood as meaning the radicals mentioned above and also, for example, the nonyloxycarbonyl, 2-methyloctyloxycarbonyl, decyloxycarbonyl or dodecyloxycarbonyl group;

the term “(C₁-C₄)-alkoxycarbonyl-(C₁-C₄)-alkyl” is to be understood as meaning, for example, a methoxycarbonylmethyl, ethoxycarbonylmethyl or methoxycarbonylethyl group;

the term “(C₁-C₄)-haloalkoxycarbonyl” is to be understood as meaning a (C₁-C₄)-alkoxycarbonyl group in which one or more, in the case of fluorine optionally also all, hydrogen atoms are replaced by halogen, preferably by fluorine or chlorine;

the term “(C₁-C₄)-alkylthio” is to be understood as meaning an alkylthio group whose hydrocarbon radical has the meaning given under the term “(C₁-C₄)-alkyl”;

the term “(C₁-C₈)-alkylthio” is to be understood as meaning an alkylthio group whose alkyl radical has the meaning given under the term “(C₁-C₈)-alkyl”;

the term “(C₁-C₄)-haloalkylthio” is to be understood as meaning a (C₁-C₄)-alkylthio group in which one or more, in the case of fluorine optionally also all, hydrogen atoms of the hydrocarbon moiety are replaced by halogen, in particular by chlorine or fluorine;

the term “(C₁-C₄)-alkylsulfinyl” is to be understood as meaning, for example, the methylsulfinyl, ethylsulfinyl, propylsulfinyl, isopropylsulfinyl, butylsulfinyl, isobutylsulfinyl, sec-butylsulfinyl or tert-butylsulfinyl group;

the term “(C₁-C₈)-alkylsulfinyl” is to be understood as meaning one of the alkylsulfinyl groups mentioned above and also, for example, the pentylsulfinyl-, 2-methylbutylsulfinyl, hexylsulfinyl or octylsulfinyl group;

the term “(C₁-C₄)-alkylsulfonyl” is to be understood as meaning, for example, the methylsulfonyl, ethylsulfonyl, propylsulfonyl, isopropylsulfonyl, butylsulfonyl, isobutylsulfonyl, sec-butylsulfonyl or tert-butylsulfonyl group;

the terms “(C₁-C₄)-haloalkylsulfinyl” and “(C₁-C₄)-haloalkylsulfonyl” are to be understood as meaning (C₁-C₄)-alkylsulfinyl and (C₁-C₄)-alkylsulfonyl radicals, respectively, having the meanings given above in which one or more, in the case of fluorine optionally also all, hydrogen atoms of the hydrocarbon moiety are replaced by halogen, in particular by chlorine or fluorine;

the terms “fluoromethylsulfinyl” and “fluoromethylsulfonyl” are to be understood as meaning the mono-, di- and trifluoromethylsulfinyl and the mono-, di- and trifluoromethylsulfonyl group, respectively;

the term “(C₁-C₄)-alkoxy” is to be understood as meaning an alkoxy group whose hydrocarbon radical has the meaning given under the term “(C₁-C₄)-alkyl”;

the term “(C₁-C₈)-alkoxy” is to be understood as meaning an alkoxy group whose hydrocarbon radical has the meaning given under the term “(C₁-C₈)-alkyl”;

the term “(C₁-C₈)-alkylsulfonyl” is to be understood as meaning one of the alkylsulfonyl groups mentioned above, and also, for example, the pentylsulfonyl, 2-methylbutylsulfonyl, hexylsulfonyl, heptylsulfonyl or octylsulfonyl group;

the term “(C₁-C₄)-alkylamino” is to be understood as meaning, for example, the methylamino, ethylamino, propylamino, isopropylamino, butylamino, isobutylamino, sec-butylamino or the tert-butylamino group;

the term “(C₁-C₈)-alkylamino” is to be understood as meaning one of the (C₁-C₄)-alkylamino groups mentioned above and also, for example, the pentylamino, hexylamino, heptylamino or octylamino group;

the term “(C₁-C₄)-dialkylamino” is to be understood as meaning, for example, the dimethylamino, methylethylamino, diethylamino, dipropylamino or the dibutylamino group, but also cyclic systems, such as, for example, the pyrrolidino or piperidino group;

the term “(C₁-C₈)-dialkylamino” is to be understood as meaning one of the (C₁-C₄)-dialkylamino groups mentioned above, and also, for example, the dipentylamino, dihexylamino or the dioctylamino group;

the term “(C₁-C₄)-haloalkoxy” is to be understood as meaning a haloalkoxy group whose halohydrocarbon radical has the meaning given under the term “(C₁-C₄)-haloalkyl”;

the term “(C₁-C₄)-alkoxy-(C₁-C₄)-alkyl” is to be understood as meaning, for example, a 1-methoxyethyl group, a 2-methoxyethyl group, a 2-ethoxyethyl group, a methoxymethyl or ethoxymethyl group, a 3-methoxypropyl group or a 4-butoxybutyl group;

the terms “(C₁-C₄)-haloalkoxy-(C₁-C₄)-alkyl”, “(C₁-C₄)-alkoxy-(C₁-C₄)-haloalkyl” and “(C₁-C₄)-haloalkoxy-(C₁-C₄)-haloalkyl” are to be understood as meaning (C₁-C₄)-alkoxy-(C₁-C₄)-alkyl radicals having the meanings given above in which one or more, in the case of fluorine optionally also all, hydrogen atoms of the hydrocarbon moieties in question are replaced by halogen, preferably by chlorine or fluorine;

the term “(C₁-C₄)-alkylthio-(C₁-C₄)-alkyl” is to be understood as meaning, for example, methylthiomethyl, ethylthiomethyl, propylthiomethyl, 2-methylthioethyl, 2-ethylthioethyl or 3-methylthiopropyl;

the term “aryl” is to be understood as meaning a carbocyclic aromatic radical having preferably 6 to 14, in particular 6 to 12, carbon atoms, such as phenyl or naphthyl, preferably phenyl;

the term “heterocyclyl” is to be understood as meaning a heteroaromatic or heteroaliphatic ring system, where “heteroaromatic ring system” is to be understood as meaning an aryl radical in which at least one CH group is replaced by N and/or at least two adjacent CH groups are replaced by S, NH or O, for example a thiophene, furan, pyrrole, thiazole, oxazole, imidazole, isothiazole, isoxazole, pyrazole, 1,3,4-oxadiazole, 1,3,4-thiadiazole, 1,3,4-triazole, 1,2,4-oxadiazole, 1,2,4-thiadiazole, 1,2,4-triazole, 1,2,3-triazole, 1,2,3,4-tetrazole, benzo[b]thiophene, benzo[b]furan, indole, benzo[c]thiophene, benzo[c]furan, isoindole, benzoxazole, benzothiazole, benzimidazole, benzisoxazole, benzisothiazole, benzopyrazole, benzothiadiazole, benzotriazole, dibenzofuran, dibenzothiophene, carbazole, pyridine, pyrazine, pyrimidine, pyridazine, 1,3,5-triazine, 1,2,4-triazine, 1,2,5-triazine, quinoline, isoquinoline, quinoxaline, quinazoline, cinnoline, 1,8-naphthyridine, 1,5-naphthyridine, 1,6-naphthyridine, 1,7-naphthyridine, phthalazine, pyridopyrimidine, purine, pteridine or 4H-quinolizine radical;

and the term “heteroaliphatic ring system” is to be understood as meaning a (C₃-C₈)-cycloalkyl radical in which at least one carbon unit is replaced by O, S or a group NR¹¹, where R¹¹ is hydrogen, (C₁-C₄)-alkyl, (C₁-C₄)-alkoxy or aryl;

the term “arylthio” is to be understood as meaning, for example, the phenylthio group;

the term “aryloxy” is to be understood as meaning, for example, the phenoxy group;

the term “heterocyclyloxy” or “heterocyclylthio” is to be understood as meaning one of the heterocyclic radicals mentioned above which is attached via an oxygen or sulfur atom;

the term “(C₃-C₈)-cycloalkoxy” or “(C₃-C₈)-cycloalkylthio” is to be understood as meaning one of the (C₃-C₈)-cycloalkyl radicals listed above which is attached via an oxygen or sulfur atom;

the term “aryl-(C₁-C₄)-alkanoyl” is to be understood as meaning, for example, the phenylacetyl, 3-phenylpropionyl, 2-phenylpropionyl, 2-methyl-2-phenylpropionyl or the 4-phenylbutyryl group;

the term “(C₃-C₈)-cycloalkyl-(C₁-C₄)-alkanoyl” is to be understood as meaning, for example, the cyclopropylcarbonyl, cyclobutylcarbonyl, cyclopentylcarbonyl, cyclohexylcarbonyl, cyclohexylacetyl or the cyclohexylbutyryl group;

the term “heterocyclyl-(C₁-C₄)-alkanoyl” is to be understood as meaning, for example, the thienoyl, furoyl, nicotinoyl, thienylacetyl or the pyridinepropionyl group;

the term “(C₃-C₈)-cycloalkoxycarbonyl” is to be understood as meaning, for example, the cyclobutyloxycarbonyl, cyclopentyloxycarbonyl, cyclohexyloxycarbonyl or the cycloheptyloxycarbonyl group;

the term “(C₃-C₈)-cycloalkyl-(C₁-C₄)-alkoxycarbonyl” is to be understood as meaning, for example, the cyclopropylmethoxycarbonyl, cyclobutylmethoxycarbonyl, cyclopentylmethoxycarbonyl, cyclohexylmethoxycarbonyl, 1-(cyclohexyl)-ethoxycarbonyl or the 2-(cyclohexyl)ethoxycarbonyl group;

the term “unsubstituted or substituted aryl, heterocyclyl, phenyl, etc.” is to be understood as meaning substitution by one or more, preferably 1 to 3, in the case of halogen also up to the maximum number, radicals selected from the group consisting of halogen, cyano, nitro, amino, hydroxyl, thio, (C₁-C₄)-alkyl, (C₁-C₄)-haloalkyl, (C₃-C₈)-cycloalkyl, (C₁-C₄)-haloalkylthio, (C₁-C₄)-alkylamino, (C₁-C₄)-haloalkylamino, formyl and (C₁-C₄)-alkanoyl.

The illustration given above applies correspondingly to homologs or radicals derived therefrom.

The invention relates to compounds of the formula (I) in the form of the free base or an acid addition salt. Acids which can be used for salt formation are, for example, inorganic acids, such as hydrochloric acid, hydrobromic acid, nitric acid, sulfuric acid or phosphoric acid, or organic acids, such as formic acid, acetic acid, propionic acid, malonic acid, oxalic acid, fumaric acid, adipic acid, stearic acid, oleic acid, methanesulfonic acid, benzenesulfonic acid or toluenesulfonic acid.

Some of the compounds of the formula (I) have one or more chiral carbon atoms or stereoisomers on double bonds. Accordingly, enantiomers or diastereomers may occur. The invention relates both to the pure isomers and to their mixtures. The mixtures of diastereomers can be separated into the components using customary methods, for example selective crystallization from suitable solvents or chromatography. Racemates can be separated by customary methods into the enantiomers, for example by salt formation with a chiral enantiomerically pure acid, separation of the diastereomeric salts and release of the pure enantiomers using a base.

The preparation of the compounds according to the invention is carried out by methods known from the literature, as described in standard works on organic synthesis, for example Houben-Weyl, Methoden der Organischen Chemie [Methods of Organic Chemistry], Georg-Thieme-Verlag, Stuttgart.

The preparation is carried out under reaction conditions which are known and suitable for the reactions mentioned. It is also possible to employ variants which are known per se but not mentioned in more detail here.

If desired, the starting materials can also be formed in situ, by not isolating them from the reaction mixture but converting them directly into the compounds of the formula (I).

To prepare a compound (I), for example, initially an ester derivative, known from the literature, of azetidine-3-carboxylic acid (A. G. Anderson, R. Lok, J. Org. Chem. 37, 1972, 3953) is reacted with a suitably substituted alkyl halide or acyl halide or, under reductive conditions, with a suitably substituted aldehyde, to give intermediate (II) and (IIa), respectively. (Scheme 1)

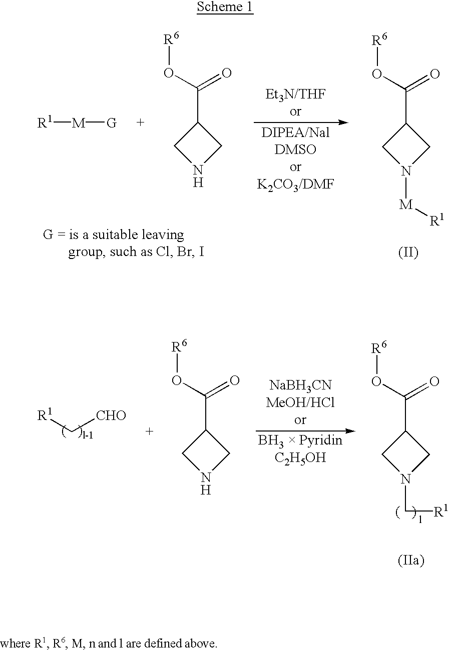

Alternatively, it is also possible to convert a suitably substituted amine after reaction with epichlorohydrin into the intermediate (III) which can be cyclized in triethylamine to give azetidine (IV). The alcohol (IV) is then, in a process known per se, converted into the substituted azetidinecarboxylic acid (IIa) (see, for example, EP-A-0 161 722) (Scheme 2). Following mesylation and reaction with the carbanion R²R³C⁻(CN), by a process known per se (for example U.S. Pat. No. 4,144,245), compounds of the formula (IV) can also be converted directly into compounds having structure (I), where X=CN and n=0.

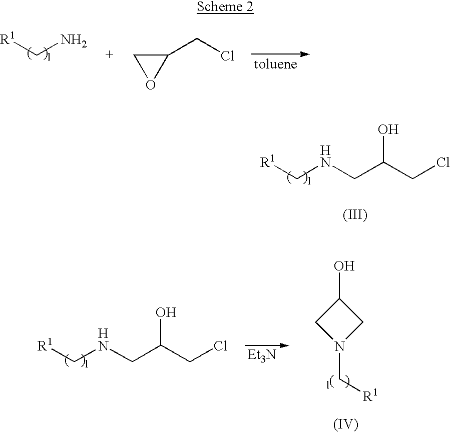

If R² equals R³, intermediate (II) is reacted, for example, with two or more equivalents of a Grignard or lithium reagent prepared from a suitably substituted aryl halide, giving the tertiary alcohol (V) (Scheme 3).

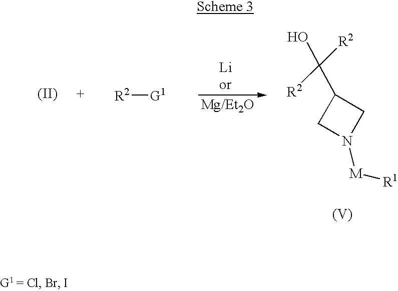

If R² does not equal R³, the azetidine-3-carbonitrile, which is initially obtained as an intermediate in the synthesis of the azetidine-3-carboxylic acid, can be alkylated or acylated by one of the methods mentioned above to give the nitrile (VI) and then be reacted with the Grignard or lithium reagent prepared from a suitably substituted aryl halide. Hydrolysis gives the ketone (VII) which can be converted by reaction with a Grignard or lithium reagent prepared from a second suitably substituted aryl halide into the desired product (Va) (Scheme 4). Alternatively, for example, the ester (II) can be converted into the N-methyl-N-methoxyamide (Weinreb amide), which can then be reacted with the appropriate Grignard or lithium reagent to give the corresponding ketone (VII).

Structures (V) and (Va) are examples of structure (I) where X=OH and n=0. Compounds having structure (I) where X=F and n=0 can be prepared from compounds (I) where X=OH and n=0 by treatment with DAST (diethylaminosulfur trifluoride) or with HF/Et₃N, according to known procedures. Compounds having structure (I) where X=H and n=0 can be prepared from compounds (I) where X=OH and n=0 after treatment with a trialkylsilane (for example Et₃SiH) and CF₃CO₂H following known procedures.

Compounds having structure (I) where Y=H and n=1 can be prepared from compounds (I) where n=0 by treatment with an acid (for example HCl, MeSO₃H) by processes known per se. Compounds having structure (I) where Y=O and n=1 can be obtained from compounds (I) where n=0 by treatment with an oxidizing agent (for example MCPBA (meta-chloroperbenzoic acid), H₂O₂), by processes known per se. Compounds having structure (I) where Y=R⁴ and n=1 can be obtained from compounds (I) where n=0 by reaction with a compound R⁴-G¹ by methods known per se. Compounds of the structure (I) where Y=OH and n=1 can be obtained from compounds (I) where Y=O and n=1 by treatment with an acid (for example HCl, MeSO₃H) by methods known per se. Compounds having the structure (I) where Y=OR⁴ and n=1 can be obtained by treatment with R⁴-G¹ following procedures known per se.

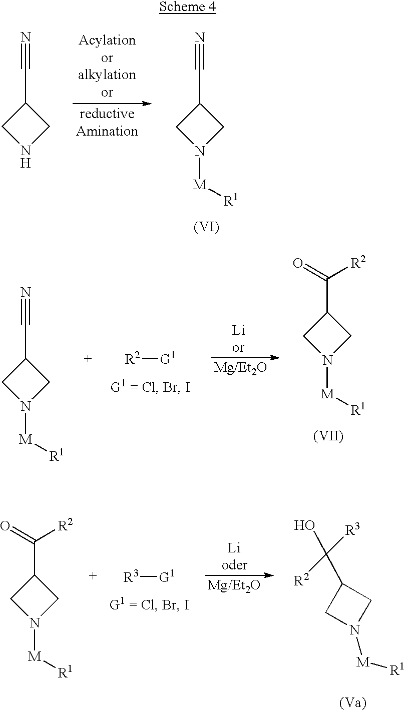

Collections of compounds of the formula (I) which can be synthesized by the abovementioned scheme may also be prepared in a parallel manner and this may be effected manually or in a semiautomated or fully automated manner. In this case, it is possible, for example, to automate the procedure of the reaction, the work-up or the purification of the products or of the intermediates. In total, this is to be understood as meaning a procedure as is described, for example, by S. H. DeWitt in “Annual Reports in Combinatorial Chemistry and Molecular Diversity: Automated synthesis”, Volume 1, Verlag Escom 1997, pages 69 to 77.

A number of commercially available apparatuses as are offered by, for example, Stem Corporation, Woodrolfe Road, Tollesbury, Essex, CM98SE, England or H+P Labortechnik GmbH, Bruckmannring 28, 85764 Oberschleiβheim, Germany, may be used for the parallel procedure of the reaction and work-up. For the parallel purification of compounds of the formula (I), or of intermediates obtained during the preparation, use may be made, inter alia, of chromatography apparatuses, for example those from ISCO, Inc., 4700 Superior Street, Lincoln, Nebr. 68504, USA.

The apparatuses mentioned lead to a modular procedure in which the individual process steps are automated, but manual operations have to be performed between the process steps. This can be avoided by employing semiintegrated or fully integrated automation systems where the automation modules in question are operated by, for example, robots. Such automation systems can be obtained, for example, from Zymark Corporation, Zymark Center, Hopkinton, Mass. 01748, USA.

In addition to what has been described here, compounds of the formula (I) may be prepared in part or fully by solid-phase-supported methods. For this purpose, individual intermediate steps or all intermediate steps of the synthesis or of a synthesis adapted to suit the procedure in question are bound to a synthetic resin. Solid-phase-supported synthesis methods are described extensively in the specialist literature, for example Barry A. Bunin in “The Combinatorial Index”, Verlag Academic Press, 1998.

The use of solid-phase-supported synthesis methods permits a series of protocols which are known from the literature and which, in turn, can be performed manually or in an automated manner. For example, the “tea-bag method” (Houghten, U.S. Pat. No. 4,631,211; Houghten et al., Proc. Natl. Acad. Sci, 1985, 82, 5131-5135), in which products from IRORI, 11149 North Torrey Pines Road, La Jolla, Calif. 92037, USA, are employed, may be semiautomated. The automation of solid-phase-supported parallel syntheses is performed successfully, for example, by apparatuses from Argonaut Technologies, Inc., 887 Industrial Road, San Carlos, Calif. 94070, USA or MultiSynTech GmbH, Wullener Feld 4, 58454 Witten, Germany.

The preparation according to the processes described herein yields compounds of the formula (I) in the form of substance collections which are referred to as libraries. The present invention also relates to libraries which comprise at least two compounds of the formula (I).

The compounds of the formula (I) are suitable for controlling animal pests, in particular insects, arachnids, helminths and molluscs, very especially preferably for controlling insects and arachnids, which are encountered in agriculture, in livestock breeding, in forests, in the protection of stored goods and materials and in the hygiene sector, and have good plant tolerance and favorable toxicity to warm-blooded species. They are active against normally sensitive and resistant species and against all or individual development stages. The abovementioned pests include:

From the order of the Acarina, for example, Acarus siro, Argas spp., Ornithodoros spp., Dermanyssus gallinae, Eriophyes ribis, Phyllocoptruta oleivora, Boophilus spp., Rhipicephalus spp., Amblyomma spp., Hyalomma spp., Ixodes spp., Psoroptes spp., Chorioptes spp., Sarcoptes spp., Tarsonemus spp., Bryobia praetiosa, Panonychus spp., Tetranychus spp., Eotetranychus spp., Oligonychus spp. and Eutetranychus spp.

From the order of the Isopoda, for example, Oniscus asselus, Armadium vulgare and Porcellio scaber.

From the order of the Diplopoda, for example, Blaniulus guttulatus.

From the order of the Chilopoda, for example, Geophilus carpophagus and Scutigera spp.

From the order of the Symphyla, for example, Scutigerella immaculata.

From the order of the Thysanura, for example, Lepisma saccharina.

From the order of the Collembola, for example, Onychiurus armatus.

From the order of the Orthoptera, for example, Blatta orientalis, Periplaneta americana, Leucophaea madeira, Blattella germanica, Acheta domesticus, Gryllotalpa spp., Locusta migratoria migratorioides, Melanoplus differentialis and Schistocerca gregaria.

From the order of the Isoptera, for example, Reticulitermes spp.

From the order of the Anoplura, for example, Phylloera vastatrix, Pemphigus spp., Pediculus humanus corporis, Haematopinus spp. and Linognathus spp.

From the order of the Mallophaga, for example, Trichodectes spp. and Damalinea spp.

From the order of the Thysanoptera, for example, Hercinothrips femoralis and Thrips tabaci.

From the order of the Heteroptera, for example, Eurygaster spp., Dysdercus intermedius, Piesma quadrata, Cimex lectularius, Rhodnius prolixus and Triatoma spp.

From the order of the Homoptera, for example, Aleurodes brassicae, Bemisia tabaci, Trialeurodes vaporariorum, Aphis gossypii, Brevicoryne brassicae, Cryptomyzus ribis, Doralis fabae, Doralis pomi, Eriosoma lanigerum, Hyalopterus arundinis, Macrosiphum avenae, Myzus spp., Phorodon humuli, Rhopalosiphum padi, Empoasca spp., Euscelus bilobatus, Nephotettix cincticeps, Lecanium corni, Saissetia oleae, Laodelphax striatellus, Nilaparvata lugens, Aonidiella aurantii, Aspidiotus hederae, Pseudococcus spp. and Psylla spp.

From the order of the Lepidoptera, for example, Pectinophora gossypiella, Bupalus piniarius, Cheimatobia brumata, Lithocolletis blancardella, Hyponomeuta padella, Plutella maculipennis, Malacosoma neustria, Euproctis chrysorrhoea, Lymantria spp., Bucculatrix thurberiella, Phyllocnistis citrella, Agrotis spp., Euxoa spp., Feltia spp., Earias insulana, Heliothis spp., Laphygma exigua, Mamestra brassicae, Panolis flammea, Prodenia litura, Spodoptera spp., Trichoplusia ni, Carpocapsa pomonella, Pieris spp., Chilo spp., Pyrausta nubilalis, Ephestia kuehniella, Galleria mellonella, Cacoecia podana, Capua reticulana, Choristoneura fumiferana, Clysia ambiguella, Homona magnanima and Tortrix viridana.

From the order of the Coleoptera, for example, Anobium punctatum, Rhizopertha dominica, Bruchidius obtectus, Acanthoscelides obtectus, Hylotrupes bajulus, Agelastica alni, Leptinotarsa decemlineata, Phaedon cochleariae, Diabrotica spp., Psylloides chrysocephala, Epilachna varivestis, Atomaria spp., Oryzaephilus surinamensis, Anthonumus spp., Sitophilus spp., Otiorrhynchus sulcatus, Cosmopolites sordidus, Ceuthorrynchus assimilis, Hypera postica, Dermestes spp., Trogoderma, Anthrenus spp., Attagenus spp., Lyctus spp., Meligethes aeneus, Ptinus spp., Niptus hololeucus, Gibbium psylloides, Tribolium spp., Tenebrio molitor, Agriotes spp., Conoderus spp., Melolontha melolontha, Amphimallon solstitialis and Costelytra zealandica.

From the order of the Hymenoptera, for example, Diprion spp., Hoplocampa spp., Lasius spp., Monomorium pharaonis and Vespa spp.

From the order of the Diptera, for example, Aedes spp., Anopheles spp., Culex spp., Drosophila melanogaster, Musca spp., Fannia spp., Calliphora erythrocephala, Lucilia spp., Chrysomyia spp., Cuterebra spp., Gastrophilus spp., Hypobosca spp., Stomoxys spp., Oestrus spp., Hypoderma spp., Tabanus spp., Tannia spp., Bibio hortulanus, Oscinella frit, Phorbia spp., Pegomyia hyoscyami, Ceratitis capitata, Dacus oleae and Tipula paludosa.

From the order of the Siphonaptera, for example, Xenopsylla cheopsis and Ceratophyllus spp.

From the order of the Arachnida, for example, Scorpio maurus and Latrodectus mactans.

From the class of helminths, for example, Haemonchus, Trichostrongulus, Ostertagia, Cooperia, Chabertia, Strongyloides, Oesophagostomum, Hyostrongulus, Ancylostoma, Ascaris and Heterakis, as well as Fasciola.

From the class of the Gastropoda, for example, Deroceras spp., Arion spp., Lymnaea spp., Galba spp., Succinea spp., Biomphalaria spp., Bulinus spp. and Oncomelania spp.

From the class of Bivalva, for example, Dreissena spp.

The phytoparasitic nematodes which can be controlled according to the invention include, for example, the root-parasitic soil nematodes, such as, for example, those of the genera Meloidogyne (root gall nematodes, such as Meloidogyne incognita, Meloidogyne hapla and Meloidogyne javanica), Heterodera and Globodera (cyst-forming nematodes, such as Globodera rostochiensis, Globodera pallida and Heterodera trifolii) and of the genera Radopholus, such as Radopholus similis, Pratylenchus, such as Pratylenchus neglectus, Pratylenchus penetrans and Pratylenchus curvitatus,

Tylenchulus, such as Tylenchulus semipenetrans, Tylenchorhynchus, such as Tylenchorhynchus dubius and Tylenchorhynchus claytoni, Rotylenchus, such as Rotylencus robustus, Heliocotylenchus, such as Heliocotylenchus multicinctus, Belonoaimus, such as Belonoaimus longicaudatus, Longidorus, such as Longidorus elongatus, Trichodorus, such as Trichodorus primitivus and Xiphinema, such as Xiphinema index.

The nematode genera Ditylenchus (stem parasites, such as Ditylenchus dipsaci and Ditylenchus destructor), Aphelenchoides (leaf nematodes, such as Aphelenchoides ritzemabosi) and Anguina (blossom nematodes, such as Anguina tritici) can furthermore be controlled with the compounds according to the invention.

The invention also relates to compositions, for example crop protection compositions, preferably insecticidal, acaricidal, ixodicidal, nematicidal, molluscidal or fungicidal, particularly preferably insecticidal and acaricidal compositions, which comprise one or more compounds of the formula (I) in addition to suitable formulation auxiliaries.

In general, the compositions according to the invention comprise from 1 to 95% by weight of the active compounds of the formula (I).

For preparing the compositions according to the invention, the active compound and the other additives are combined and formulated as a suitable use form.

Wettable powders (WP), emulsifiable concentrates (EC), aqueous solutions (SL), emulsions, sprayable solutions, oil- or water-based dispersions (SC), suspoemulsions (SE), dusting powders (DP), seed dressings, granules in the form of microgranules, sprayed granules, absorption granules and adsorption granules, water-dispersible granules (WG), ULV formulations, microcapsules, waxes or baits. In addition, the compositions can be employed as dips or mist applications, in the form of foams, pastes, gels, ointments, lotions, shampoos, hair-setting compositions, active-compound-containing mats (for example flat or in the form of a cushion), impregnated articles, aerosols, pressurized and non-pressurized sprays, additives to color lacques and foodstuff, and also for use as fumigants and evaporator compositions, as combustible solids (for example in the form of a cone or coil) or as combustible oils (distributed, for example, via a heated wick) and in further formulations familiar to the person skilled in the art.

These individual types of formulation are known in principle and are described, for example, in: Winnacker-Küchler, “Chemische Technologie” [Chemical Technology], Volume 7, C. HanserVerlag Munich, 4th Edition 1986; van Falkenberg, “Pesticides Formulations”, Marcel Dekker N.Y., 2nd Edition 1972-73; K. Martens, “Spray Drying Handbook”, 3rd Edition 1979, G. Goodwin Ltd. London.

The necessary formulation auxiliaries, such as inert materials, surfactants, solvents and further additives, are likewise known and are described, for example, in: Watkins, “Handbook of Insecticide Dust Diluents and Carriers”, 2nd Edition, Darland Books, Caldwell N.J.; H. v. Olphen, “Introduction to Clay Colloid Chemistry”, 2nd Edition, J. Wiley & Sons, N.Y.; Marsden, “Solvents Guide”, 2nd Edition, Interscience, N.Y. 1950; McCutcheon's, “Detergents and Emulsifiers Annual”, MC Publ. Corp., Ridgewood N.J.; Sisley and Wood, “Encyclopedia of Surface Active Agents”, Chem. Publ. Co. Inc., N.Y. 1964, Schönfeldt, “Grenzflächenaktive Äthylenoxidaddukte” [Surface-active ethylene oxide adducts], Wiss. Verlagsgesell., Stuttgart 1967; Winnacker-Küchler, “Chemische Technologie” [Chemical Technology], Volume 7, C. Hanser Verlag Munich, 4th Edition 1986.

Combinations with other substances having a pesticidal action, fertilizers and/or growth regulators can be prepared on the basis of these formulations, for example in the form of a ready-to-use formulation or as a tank mix. Wettable powders are preparations which are uniformly dispersible in water and which, alongside the active compound, and in addition to a diluent or inert substance, also comprise wetting agents, for example polyethoxylated alkylphenols, polyethoxylated fatty alcohols or alkyl- or alkylphenolsulfonates, and dispersing agents, for example sodium ligninsulfonate or sodium 2,2′-dinaphthylmethane-6,6′-disulfonate.

Emulsifiable concentrates are prepared by dissolving the active compound in an organic solvent, for example butanol, cyclohexanone, dimethylformamide, xylene or also higher-boiling aromatics or hydrocarbons, with the addition of one or more emulsifiers. Emulsifiers which can be used are, for example: calcium alkylaryl-sulfonates, such as Ca dodecylbenzenesulfonate, or nonionic emulsifiers, such as fatty acid polyglycol esters, alkylaryl polyglycol ethers, fatty alcohol polyglycol ethers, propylene oxide/ethylene oxide condensation products, alkyl polyethers, sorbitan fatty acid esters, polyoxyethylene sorbitan fatty acid esters or polyoxyethylene sorbitol esters.

Dusting powders are obtained by grinding the active compound with finely divided solid substances, for example talc, naturally occurring clays, such as kaolin, bentonite and pyrophyllite, or diatomaceous earth. Granules can be prepared either by spraying the active compound onto granular inert material capable of adsorption or by applying active compound concentrates to the surface of carrier substances, such as sand, kaolinites or granular inert material, by means of adhesives, for example polyvinyl alcohol, sodium polyacrylate or mineral oils. Suitable active compounds can also be granulated in the manner customary for the preparation of fertilizer granules—if desired as a mixture with fertilizers.

Aerosols, sold, for example, in cans, are prepared by dissolving the active compound in water and/or organic solvents, such as, for example, acetone, deodorized petroleum, saturated C₈-C₁₃-hydrocarbons, vegetable oils, with addition of further suitable substances, such as, for example, emulsifiers, piperonyl butoxide, sorbitan monooleate, polyoxyethylene glycerol monooleate, fragrances and suitable propellents, such as, for example, carbon dioxide or butane. Ready-to-use sprays, for example for use in rooms, are obtained, for example, by mixing the active compound with odorless kerosine and antioxidants, it being possible to admix further additives, such as, for example, emulsifiers, synergists (for example piperonyl butoxide) or fragrances. Baits can be prepared, for example, by mixing the active compound with attractants and/or foodstuffs, such as, for example, sugar, and also carrier materials, such as, for example, paraffin wax.

A further advantageous embodiment for use in rooms is the use as a fumigant and an evaporator composition, which can be employed by various methods. In one of these methods, combustible solids, such as, for example, sawdust (for example pine sawdust), starch and coconut shell powder and also powdered leaves and stalks of further plants (for example pyrethrum, cedar) are, with addition of colorants and, if appropriate, fungicides, solidifed in specific forms, such as, for example, a meander, a coil or a cone, using suitable binders, and the active compound is then applied. The active compound is then distributed in the room by slow and controlled burning. In another method, mats or cushions of non-combustible fibers are used as carriers into which the active compound and, if appropriate, further substances are incorporated. These carriers are placed onto a heating plate which is heated under controlled conditions, thus releasing the active compound. In a further method, an oil is used to which the active compound is added and into which a wick consisting, for example, of cotton and/or cellulose in compressed form, is dipped, which wick releases the active compound from the oil into the room on burning. In a variant of this method, a wick of non-combustible fibers is used which is heated by an electric device, thus effecting the distribution of the active compound contained in the oil throughout the room. In the above-mentioned methods, the active compound is applied either directly or in already formulated form. Frequently, for example, colorants and fragrances are added, and also suitable fungicides for protecting the carriers made of natural products against natural decomposition.

In wettable powders, the active compound concentration is usually about 10 to 90% by weight, the remainder to make up 100% by weight comprising customary formulation constituents. In emulsifiable concentrates, the active compound concentration can be about 5 to 80% by weight. Dust-like formulations usually comprise 5 to 20% by weight of active compound, and sprayable solutions about 2 to 20% by weight. In granules, the content of active compound partly depends on whether the active compound is present in liquid or solid form and what granulating auxiliaries, fillers and the like are used. Baits generally comprise from 0.01 to 60% by weight of active compound, preferably from 0.1 to 5% by weight; aerosols generally comprise from 0.01 to 50% by weight, preferably from 0.1 to 5% by weight; ready-to-use sprays generally comprise from 0.01 to 50% by weight, preferably from 0.05 to 10% by weight. The active compound contents in fumigants and evaporator compositions are, in the case of combustible solids, generally in the range from 0.01 to 60% by weight, in the case of active-compound-comprising mats and cushions in the range from 0.01 to 60% by weight and in the case of active-compound-comprising oils in the range from 0.01 to 90% by weight.

In addition, the active compound formulations mentioned comprise, if appropriate, the particular customary tackifiers, wetting agents, dispersing agents, emulsifiers, penetration agents, solvents, fillers or carrier substances.

For use, the concentrates in the commercially available form are diluted in the customary manner, if appropriate, for example by means of water in the case of wettable powders, emulsifiable concentrates, dispersions and in some cases also microgranules. Dust-like and granular formulations as well as sprayable solutions are usually not diluted further with additional inert substances before use.

The required amount applied varies with the external conditions, such as temperature or humidity. It can vary within wide limits, for example between 0.0005 and 10.0 kg/ha or more of active substance, but is preferably between 0.001 and 5 kg/ha.

The active compounds according to the invention can be present in their commercially available formulations and in the use forms prepared from these formulations (see the above mentioned compositions) as mixtures with other active compounds, such as insecticides, attractants, sterilizing agents, acaricides, nematicides, fungicides, molluscides, growth-regulating substances or herbicides. The pesticides include, for example, phosphoric acid esters, carbamates, carboxylic acid esters, formamidines, tin compounds and substances produced by micro-organisms.

Preferred partners for the mixtures are:

1. from the group of phosphorus compounds

acephate, azamethiphos, azinphos-ethyl, azinphos-methyl, bromophos, bromophos-ethyl, cadusafos (F-67825), chlorethoxyphos, chlorfenvinphos, chlormephos, chlorpyrifos, chlorpyrifos-methyl, demeton, demeton-S-methyl, demeton-S-methyl sulfone, dialifos, diazinon, dichlorvos, dicrotophos, dimethoate, disulfoton, EPN, ethion, ethoprophos, etrimfos, famphur, fenamiphos, fenitriothion, fensulfothion, fenthion, fonofos, formothion, fosthiazate (ASC-66824), heptenophos, isazophos, isothioate, isoxathion, malathion, methacrifos, methamidophos, methidathion, salithion, mevinphos, monocrotophos, naled, omethoate, oxydemeton-methyl, parathion, parathion-methyl, phenthoate, phorate, phosalone, phosfolan, phosphocarb (BAS-301), phosmet, phosphamidon, phoxim, pirimiphos, primiphos-ethyl, pirimiphos-methyl, profenofos, propaphos, proetamphos, prothiofos, pyraclofos, pyridapenthion, quinalphos, sulprofos, temephos, terbufos, tebupirimfos, tetrachlorvinphos, thiometon, triazophos, trichlorphon, vamidothion;

2. from the group of carbamates

alanycarb (OK-135), aldicarb, 2-sec-butylphenyl methylcarbamate (BPMC), carbaryl, carbofuran, carbosulfan, cloethocarb, benfuracarb, ethiofencarb, furathiocarb, HCN-801, isoprocarb, methomyl, 5-methyl-m-cumenyl butyryl(methyl)carbamate, oxamyl, pirimicarb, propoxur, thiodicarb, thiofanox, 1-methylthio(ethylideneamino) N-methyl-N-(morpholinothio)carbamate (UC 51717), triazamate;

3. from the group of carboxylic acid esters

acrinathrin, allethrin, alphametrin, 5-benzyl-3-furylmethyl (E)-(1R)-cis-2,2-di-methyl-3-(2-oxothiolan-3-ylidenemethyl)cyclopropanecarboxylate, beta-cyfluthrin, beta-cypermethrin, bioallethrin, bioallethrin ((S)-cyclopentyl isomer), bioresmethrin, bifenthrin, (RS)-1-cyano-1-(6-phenoxy-2-pyridyl)methyl (1RS)-trans-3-(4-tert-butylphenyl)-2,2-dimethylcyclopropanecarboxylate (NCI 85193), cycloprothrin, cyfluthrin, cyhalothrin, cythithrin, cypermethrin, cyphenothrin, deltamethrin, empenthrin, esfenvalerate, fenfluthrin, fenpropathrin, fenvalerate, flucythrinate, flumethrin, fluvalinate (D isomer), imiprothrin (S-41311), lambda-cyhalothrin, permethrin, pheothrin ((R) isomer), prallethrin, pyrethrins (natural products), resmethrin, tefluthrin, tetramethrin, theta-cypermethrin (TD-2344), tralomethrin, transfluthrin and zeta-cypermethrin (F-56701);

4. from the group of amidines amitraz, chlordimeform;

5. from the group of tin compounds cyhexatin, fenbutatin oxide;

6. others

abamectin, ABG-9008, acetamiprid, Anagrapha falcitera, AKD-1022, AKD-3059, ANS-118, Bacillus thuringiensis, Beauveria bassianea, bensultap, bifenazate (D-2341), binapacryl, BJL-932, bromopropylate, BTG-504, BTG-505, buprofezin, camphechlor, cartap, chlorobenzilate, chlorfenapyr, chlorfluazuron, 2-(4-chlorophenyl)-4,5-diphenylthiophene (UBI-T 930), chlorfentezine, chromafenozide (ANS-118), CG-216, CG-217, CG-234, A-184699, 2-naphthylmethyl cyclopropanecarboxylate (Ro12-0470), cyromazin, diacloden (thiamethoxam), diafenthiuron, N-(3,5-dichloro-4-(1,1,2,3,3,3-hexafluoro-1-propyloxy)phenyl)carbamoyl)-2-chlorobenzocarboximide acid ethyl ester, DDT, dicofol, diflubenzuron, N-(2,3-dihydro-3-methyl-1,3-thiazol-2-ylidene)-2,4-xylidine, dinobuton, dinocap, diofenolan, DPX-062, emamectin-benzoate (MK-244), endosulfan, ethiprole (sulfethiprole), ethofenprox, etoxazole (YI-5301), fenazaquin, fenoxycarb, fipronil, flonicamid, fluazuron, flumite (flufenzine, SZI-121), 2-fluoro-5-(4-(4-ethoxyphenyl)-4-methyl-1-pentyl)diphenyl ether (MTI 800), granulosis and nuclear polyhedrosis viruses, fenpyroximate, fenthiocarb, flubenzimine, flucycloxuron, flufenoxuron, flufenprox (ICI-A5683), fluproxyfen, gamma-HCH, halofenozide (RH-0345), halofenprox (MTI-732), hexaflumuron (DE_{—}473), hexythiazox, HOI-9004, hydramethylnon (AC 217300), lufenuron, imidacloprid, indoxacarb (DPX-MP062), kanemite (AKD-2023), M-020, MTI-446, ivermectin, M-020, methoxyfenozide (Intrepid, RH-2485), milbemectin, NC-196, neemgard, nitenpyram (T1-304), 2-nitromethyl-4,5-dihydro-6H-thiazine (DS 52618), 2-nitromethyl-3,4-dihydrothiazole (SD 35651), 2-nitromethylene-1,2-thiazinan-3-ylcarbamaldehyde (WL 108477), pyriproxyfen (S-71639), NC-196, NC-1111, NNI-9768, novaluron (MCW-275), OK-9701, OK-9601, OK-9602, propargite, pymethrozine, pyridaben, pyrimidifen (SU-8801), RH-0345, RH-2485, RYI-210, S-1283, S-1833, SB7242, SI-8601, silafluofen, silomadine (CG-177), spinosad, SU-9118, tebufenozide, tebufenpyrad (MK-239), teflubenzuron, tetradifon, tetrasul, thiacloprid, thiocyclam, TI-435, tolfenpyrad (OMI-88), triazamate (RH-7988), triflumuron, verbutin, vertalec (Mykotal), YI-5301.

The active compound content of the use forms prepared from the commercially available formulations can be from 0.00000001 to 95% by weight of active compound, preferably between 0.00001 and 1% by weight. The active compounds are used in a customary manner appropriate for the use forms.

The invention also provides a method for controlling harmful insects, Acarina, molluscs and/or nematodes, in which an effective amount of a compound according to the invention or a composition according to the invention is applied to these organisms or the plants, areas or substrates infested with them.

The invention also provides the use of a compound according to the invention or a composition according to the invention for controlling harmful insects, Acarina, molluscs and/or nematodes.

The active substances according to the invention are also suitable for the field of veterinary medicine, preferably for controlling endo- and ectoparasites, and for the field of animal husbandry.

The active substances according to the invention can preferably be applied in a known manner, such as by oral application in the form of, for example, tablets, capsules, potions or granules, by dermal application in the form of, for example, dipping, spraying, pouring-on and spotting-on and dusting, and also by parenteral application in the form of, for example, injection.

The compounds of the formula (I) according to the invention can accordingly also be employed particularly advantageously in livestock husbandry (for example cattle, sheep, pigs and poultry such as chickens, geese etc.). In a preferred embodiment of the invention, the novel compounds, if appropriate in suitable formulations (cf. above) and if appropriate with the drinking water or feed, are administered orally to the animals. Since excretion in the feces occurs in an effective fashion, the development of insects in the animal feces can be prevented very simply in this fashion. The dosages and formulations suitable in each case, in particular, depend on the type and developmental stage of the productive animals and also on the severity of infestation and can easily be determined and fixed by conventional methods. In the case of cattle, the compounds can be employed, for example, in dosages of 0.01 to 1 mg/kg of body weight.

In addition, the compounds according to the invention are also suitable for use in industrial fields, for example as wood preservative, as preservative in paints, in cooling lubricants for metal working or as preservative in drilling and cutting oils.

Compounds of the formula (I) in their commercially available formulations can be used either alone or in combination with other fungicides known from the literature.

Examples of fungicides which are known from the literature and which can be combined in accordance with the invention with the compounds of the formula (I) are the following products:

aldimorph, andoprim, anilazine, BAS 480F, BAS 450F, benalaxyl, benodanil, benomyl, binapacryl, bitertanol, bromuconazole, buthiobate, captafol, captan, carbendazim, carboxin, CGA 173506, cyprofuram, dichlofluanid, dichlomezin, diclobutrazol, diethofencarb, difenconazole (CGA 169374), difluconazole, dimethirimol, dimethomorph, diniconazole, dinocap, dithianon, dodemorph, dodine, edifenfos, ethirimol, etridiazol, fenarimol, fenfuram, fenpiclonil, fenpropidin, fenpropimorph, fentin acetate, fentin hydroxide, ferimzone (TF164), fluazinam, fluobenzimine, fluquinconazole, fluorimide, flusilazole, flutolanil, flutriafol, folpet, fosetyl-aluminum, fuberidazole, fulsulfamide (MT-F 651), furalaxyl, furconazole, furmecyclox, guazatine, hexaconazole, ICI A5504, imazalil, imibenconazole, iprobenfos, iprodione, isoprothiolane, KNF 317, copper compounds such as copper oxychloride, oxine-copper, copper oxide, mancozeb, maneb, mepanipyrim (KIF 3535), metconazole, mepronil, metalaxyl, methasulfocarb, methfuroxam, MON 24000, myclobutanil, nabam, nitrothalidopropyl, nuarimol, ofurace, oxadixyl, oxycarboxin, penconazole, pencycuron, PP 969, probenazole, propineb, prochloraz, procymidon, propamocarb, propiconazole, prothiocarb, pyracarbolid, pyrazophos, pyrifenox, pyroquilon, rabenzazole, RH7592, sulfur, tebuconazole, TF 167, thiabendazole, thicyofen, thiofanate-methyl, thiram, tolclofos-methyl, tolylfluanid, triadimefon, triadimenol, tricyclazole, tridemorph, triflumizole, triforine, validamycin, vinchlozolin, XRD 563, zineb, sodium dodecylsulfonate, sodium dodecyl sulfate, sodium C13/C15-alcohol ether sulfonate, sodium cetostearyl phosphate ester, sodium dioctylsulfosuccinate, sodium isopropylnaphthalenesulfonate, sodium methylenebisnaphthalenesulfonate, cetyltrimethylammonium chloride, salts of long-chain primary, secondary or tertiary amines, alkylpropyleneamines, laurylpyrimidinium bromide, ethoxylated quaternized fatty amines, alkyldimethylbenzylammonium chloride and 1-hydroxylethyl-2-alkylimidazoline.

The abovementioned components are known active substances, many of which are described in C. D. S. Tomlin, S. B. Walker, The Pesticide Manual, 12th Edition, British Crop Protection Council, Farnham 2000.

The use in economically important crops of useful plants and ornamentals, for example cereals such as wheat, barley, rye, oats, millet, rice, cassaya and corn, or else in crops of sugar beet, cotton, soya, oilseed rape, potatoes, tomatoes, peas and other vegetables is preferred.

The invention also provides seed, treated or coated with an effective amount of a compound according to the invention or of a composition according to the invention.

The compounds of the formula (I) can also be employed for controlling harmful organisms in crops of known or genetically engineered plants yet to be developed. As a rule, the transgenic plants are distinguished by particular advantageous properties, for example by resistances to certain crop protection agents, resistances to plant diseases or pathogens of plant diseases such as certain insects or microorganisms such as fungi, bacteria or viruses. Other particular properties relate, for example, to the harvested material with regard to quantity, quality, storage properties, composition and specific constituents. Thus, transgenic plants with an elevated starch content or altered starch quality, or those with a different fatty acid composition of the harvested material, are known.

The use in economically important transgenic crops of useful plants and ornamentals, for example, cereals such as wheat, barley, rye, oats, millet, rice, cassaya and corn or else crops of sugar beet, cotton, soya, oilseed rape, potatoes, tomatoes, peas and other vegetables is preferred.

When being used in transgenic crops, in particular those in which the plants express an insecticide, effects are frequently found (in addition to the pesticidal effects which can be observed in other crops)-which are specific to application in the transgenic crop in question, for example an altered or specifically widened spectrum of pests which can be controlled, or altered application rates which can be used for application.

The invention therefore also provides the use of compounds of the formula (I) for controlling harmful organisms in transgenic crop plants.

The compounds of the formula (I) are also suitable for controlling animal pests, in particular arthropods, such as insects and Acarina, in rooms, specifically for controlling flies, such as, for example, from the family Muscidae (for example common house-flies, domestic flies), Calliphoridae (for example greenbottles, “death flies” (Cynomyia mortuorum), bluebottles) and Sarcophagidae (for example flesh-flies), mosquitoes, such as, for example, Aedes aegypti, Anopheles arabiensis and Culex quinquefasciatus, and cockroaches, such as, for example Blattella germanica and Periplaneta americana.

Further preferred areas of use are the protection of stored products and materials, the hygiene sector and the domestic sector where, in a preferred embodiment of the invention, the composition according to the invention is used in the buildings in question and, if appropriate, combined with further measures, such as, for example, sticky boards or traps. Here, too, suitable dosages and formulations depend in particular on the type and the intensity of the risk of infestation and can be established and determined readily by customary methods.

The use according to the invention of compounds of the formula (I) or compositions comprising them, for example as insecticide, acaricide, molluscide or nematicide, includes the case where the compound of the formula (I) or its salt is formed from a precursory substance only after application, for example in the insect, in a plant or in the soil.

The content of German Patent Application 10144872.4, whose priority the present application claims, and the appended abstracts is incorporated herein by reference.

The examples which follow serve to illustrate the invention without restricting it thereto.

### A. PREPARATION EXAMPLES

### Example 1

### Preparation of

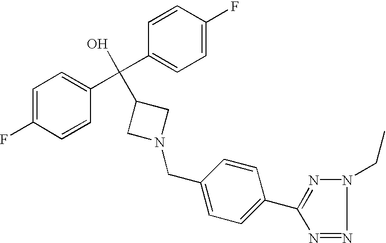

A solution of 1.4 g of 1-bromo-4-fluorobenzene in 5 ml of diethyl ether was rapidly added dropwise to 200 mg of magnesium turnings in 5 ml of diethyl ether, such that the mixture boiled gently under reflux. After the addition had ended, the mixture was boiled at reflux for 30 min and then cooled to room temperature, and 400 mg of ester A (see below) in 15 ml of dry THF were added dropwise. After the addition had ended, the mixture was boiled at reflux for 2 h and then stirred at room temperature for another 12 h. Saturated ammonium chloride solution was added, and the reaction mixture was extracted with ethyl acetate. The extracts were washed with sodium chloride solution and dried over magnesium sulfate. Following concentration under reduced pressure, the residue was purified by silica gel chromatography using ethyl acetate/pentane 1:1, which gave a yellow oil.

a) Preparation of Ester (A)

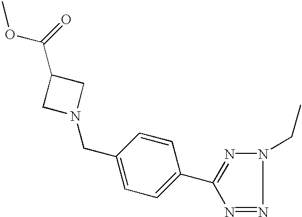

151 mg of methyl azetidine-3-carboxylate hydrochloride, 202 mg of 4-(2-ethyl-2H-tetrazol-5-yl)benzaldehyde, 200 mg of 3 A molecular sieve and 63 mg of sodium cyanoborohydride were taken up in 2 ml of anhydrous methanol and stirred at room temperature for 12 h. The mixture was adjusted to pH 10 using potassium hydroxide and extracted with diethyl ether. The organic extracts were washed with sodium chloride solution, dried over magnesium sulfate and concentrated under reduced pressure. The residue was chromatographed on silica gel using ethyl acetate/pentane 1:1, which gave a yellow oil.

### Example 2

### Preparation of

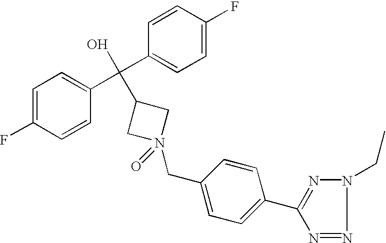

0.46 g of the compound from Example 1 in 15 ml of methanol was reacted at room temperature with 4 ml of 30% strength hydrogen peroxide for 48 h. Ethyl acetate was added to the reaction mixture, and the mixture was washed twice with water and once with 10% strength sodium thiosulfate solution. The organic phase was dried over magnesium sulfate and concentrated under reduced pressure. The residue was chromatographed on silica gel using methyl chloride/methanol 91, which gave a white solid.

### B. CHEMICAL EXAMPLES

TABLE 1
Compounds of the formula (I.1)

| | |
|---|---|
| | (I.1) |
| 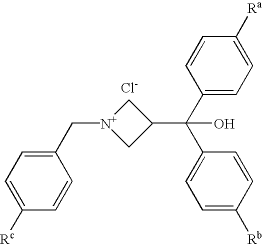 | |

| Ex. No. | R^{a} | R^{b} | R^{c} | Phys. data |
|---|---|---|---|---|
| 1.1 | OCF₃ | OCF₃ | n-propoxy | |
| 1.2 | OCF₃ | OCF₃ | OMe | |
| 1.3 | OCF₃ | OCF₃ | benzyloxy | |
| 1.4 | OCF₃ | OCF₃ | cyclopentyloxy | |
| 1.5 | OCF₃ | CF₃ | i-propyloxy | |
| 1.6 | OCF₃ | OMe | i-propyloxy | |
| 1.7 | OCF₃ | F | i-propyloxy | |
| 1.8 | CF₃ | OMe | —O—CH₂-c-prop. | |
| 1.9 | OCF₃ | CF₃ | 2-pyridyloxy | |
| 1.10 | OCF₃ | OCF₃ | NHCOOEt | |
| 1.11 | CF₃ | CF₃ | NHCOOEt | |
| 1.12 | CF₃ | F | NHCOOEt | |
| 1.13 | OCF₃ | OMe | NHCOOEt | |
| 1.14 | OCF₃ | OCF₃ | NHCOO-i-prop. | |
| 1.15 | CF₃ | CF₃ | NHCOO-i-prop | |
| 1.16 | CF₃ | CF₃ | 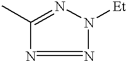 | |
| 1.17 | Cl | Cl | NHCOO-i-butyl | |
| 1.18 | CF₃ | CF₃ | NHCOO-i-butyl | |
| 1.19 | OCF₃ | OCF₃ | 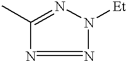 | |
| 1.20 | OCF₃ | CF₃ | 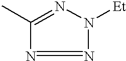 | |
| 1.21 | CF₃ | CF₃ | 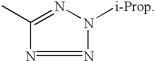 | |
| 1.22 | CF₃ | CF₃ | 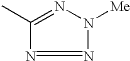 | |
| 1.23 | CF₃ | CF₃ | 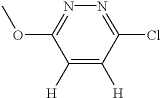 | |
| 1.24 | t-But | t-But | NHCOO-i-prop. | |
| 1.25 | CF₃ | CF₃ | 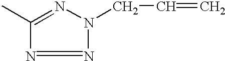 | |
| 1.26 | CF₃ | CF₃ | 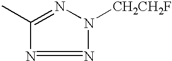 | |
| 1.27 | OCF₃ | CF₃ | 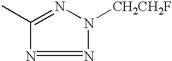 | |
| 1.28 | OCF₃ | OCF₃ | 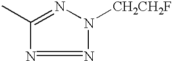 | |
| 1.29 | CF₃ | CF₃ | —C₆H₅ | |
| 1.30 | CF₃ | CF₃ | 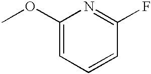 | |
| 1.31 | OCF₃ | OCF₃ | 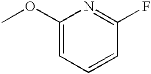 | |
| 1.32 | CF₃ | | 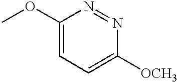 | |
| 1.33 | OCF₃ | OCF₃ | OCF₃ | |
| 1.34 | CF₃ | CF₃ | OCF₃ | |
| 1.35 | CF₃ | CF₃ | Ophenyl | |

TABLE 2
Compounds of the formula (I.2)

| | |
|---|---|
| | (I.2) |
| 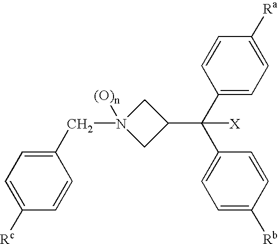 | |

| Ex. No. | R^{a} | R^{b} | R^{c} | X | n | Phys. data |
|---|---|---|---|---|---|---|
| 2.1 | OCF₃ | OCF₃ | n-propoxy | OH | 0 | |
| 2.2 | OCF₃ | OCF₃ | OEt | OH | 0 | oil |
| 2.3 | OCF₃ | OCF₃ | n-butoxy | OH | 0 | |
| 2.4 | OCF₃ | OCF₃ | OMe | OH | 0 | oil |
| 2.5 | OCF₃ | OCF₃ | n-propoxy | OH | 1 | oil |
| 2.6 | OCF₃ | OCF₃ | —O—CH₂-c-prop. | OH | 0 | |
| 2.7 | OCF₃ | OCF₃ | allyloxy | OH | 0 | |
| 2.8 | OCF₃ | OCIF₃ | i-butyloxy | OH | 0 | |
| 2.9 | OCF₃ | OCF₃ | propargyloxy | OH | 0 | |
| 2.10 | OCF₃ | OCF₃ | benzyloxy | OH | 0 | |
| 2.11 | OCF₃ | OCF₃ | s-butyloxy | OH | 0 | |
| 2.12 | OCF₃ | OCF₃ | i-propoxy | OH | 0 | |
| 2.13 | OCF₃ | OCF₃ | F | OH | 0 | |
| 2.14 | CF₃ | CF₃ | n-propoxy | OH | 0 | oil |
| 2.15 | F | OMe | n-propoxy | OH | 0 | |
| 2.16 | OCF₃ | OCF₃ | cyclopentyloxy | OH | 0 | |
| 2.17 | OCF₃ | OCF₃ | n-propoxy | OH | 0 | |
| 2.18 | OCF₃ | F | n-propoxy | OH | 0 | |
| 2.19 | CF₃ | OMe | n-propoxy | OH | 0 | |
| 2.20 | OCF₃ | OMe | OEt | OH | 0 | |
| 2.21 | OCF₃ | F | OEt | OH | 0 | |
| 2.22 | OCF₃ | F | n-butoxy | OH | 0 | |
| 2.23 | CF₃ | CF₃ | OEt | OH | 0 | |
| 2.24 | CF₃ | CF₃ | n-butoxy | OH | 0 | |
| 2.25 | OCF₃ | OMe | n-butoxy | OH | 0 | |
| 2.26 | OCF₃ | CF₃ | n-butoxy | OH | 0 | |
| 2.27 | OCF₃ | CF₃ | OEt | OH | 0 | oil |
| 2.28 | CF₃ | OCF₃ | —O—CH₂-c-prop. | OH | 0 | |
| 2.29 | OCF₃ | F | —O—CH₂-c-prop. | OH | 0 | |
| 2.30 | OCF₃ | CF₃ | —O—CH₂-c-prop. | OH | 0 | |
| 2.31 | OCF₃ | OMe | —O—CH₂-c-prop. | OH | 0 | |
| 2.32 | OCF₃ | OMe | allyloxy | OH | 0 | |
| 2.33 | OCF₃ | CF₃ | allyloxy | OH | 0 | |
| 2.34 | OCF₃ | F | i-propyloxy | OH | 0 | |
| 2.35 | OCF₃ | OMe | i-propyloxy | OH | 0 | |
| 2.36 | OCF₃ | CF₃ | i-propyloxy | OH | 0 | |
| 2.37 | OCF₃ | CF₃ | propargyloxy | OH | 0 | |
| 2.38 | OCF₃ | OMe | propargyloxy | OH | 0 | |
| 2.39 | OCF₃ | F | propargyloxy | OH | 0 | |
| 2.40 | OCF₃ | F | allyloxy | OH | 0 | |
| 2.41 | CF₃ | OMe | n-butoxy | OH | 0 | |
| 2.42 | CF₃ | OMe | OEt | OH | 0 | |
| 2.43 | OCF₃ | OCF₃ | F | OH | 1 | |
| 2.44 | CF₃ | OMe | allyloxy | OH | 0 | |
| 2.45 | CF₃ | OMe | propargyloxy | OH | 0 | |
| 2.46 | CF₃ | OMe | —O—CH₂-c-prop. | OH | 0 | |
| 2.47 | OCF₃ | OCF₃ | propargyloxy | OH | 1 | |
| 2.48 | OCF₃ | CF₃ | allyloxy | OH | 1 | |
| 2.49 | CF₃ | CF₃ | n-propoxy | OH | 1 | |
| 2.50 | OCF₃ | CF₃ | n-butoxy | OH | 1 | |
| 2.51 | CF₃ | CF₃ | n-butoxy | OH | 1 | |
| 2.52 | CF₃ | CF₃ | OEt | OH | 1 | |
| 2.53 | OCF₃ | CF₃ | —O—CH₂-c-prop. | OH | 1 | |
| 2.54 | CF₃ | CF₃ | —O—CH₂-c-prop. | OH | 1 | |
| 2.55 | OCF₃ | OW;s | cyclopentyloxy | OH | 1 | |
| 2.56 | CF₃ | F | n-propoxy | OH | 0 | |
| 2.57 | CF₃ | F | n-butoxy | OH | 0 | |
| 2.58 | CF₃ | F | —O—CH₂-c-prop. | OH | 0 | |
| 2.59 | CF₃ | F | allyloxy | OH | 0 | |
| 2.60 | CF₃ | F | propargyloxy | OH | 0 | |
| 2.61 | CF₃ | F | propargyloxy | OH | 1 | |
| 2.62 | CF₃ | F | n-propoxy | OH | 1 | |
| 2.63 | CF₃ | F | —O—CH₂-c-prop. | OH | 1 | |
| 2.64 | OCF₃ | F | —O—CH₂-c-prop. | OH | 1 | |
| 2.65 | OCF₃ | OCF₃ | 2-pyridyloxy | OH | 0 | |
| 2.66 | CF₃ | OMe | 2-pyridyloxy | OH | 0 | |
| 2.67 | OCF₃ | CF₃ | 2-pyridyloxy | OH | 0 | |
| 2.68 | OCF₃ | F | 2-pyridyloxy | OH | 0 | |
| 2.69 | OCF₃ | OCF₃ | NHCOOMe | OH | 0 | |
| 2.70 | OCF₃ | CF₃ | NHCOOMe | OH | 0 | |
| 2.71 | OCF₃ | OMe | 2-pyridyloxy | OH | 0 | |
| 2.72 | CF₃ | F | 2-pyridyloxy | OH | 0 | |
| 2.73 | CF₃ | CF₃ | 2-pyridyloxy | OH | 0 | |
| 2.74 | CF₃ | CF₃ | NHCOOMe | OH | 0 | |
| 2.75 | CF₃ | F | NHCOOMe | OH | 0 | |
| 2.76 | OCF₃ | OMe | NHCOOMe | OH | 0 | |
| 2.77 | OCF₃ | CF₃ | NHCOOMe | OH | 1 | |
| 2.78 | OCF₃ | OCF₃ | NHCOOMe | OH | 1 | |
| 2.79 | OCF₃ | OCF₃ | NHCOOEt | OH | 0 | |
| 2.80 | OCF₃ | OMe | NHCOOR | OH | 0 | |
| 2.81 | OCF₃ | CF₃ | NHCOOEt | OH | 0 | |
| 2.82 | CF₃ | CF₃ | NHCOM | OH | 0 | |
| 2.83 | CF₃ | F | NHCOOEt | OH | 0 | |
| 2.84 | OCF₃ | OCF₃ | NHCOOEt | OH | 1 | |
| 2.85 | OCF₃ | CF₃ | NHCOOEt | OH | 1 | |
| 2.86 | CF₃ | CF₃ | NHCOOEt | OH | 1 | |
| 2.87 | CF₃ | F | NHCOOEt | OH | 1 | |
| 2.88 | OCF₃ | OCF₃ | NHCOOMe | H | 0 | |
| 2.89 | OCF₃ | OCF₃ | NHCOOMe | H | 1 | |
| 2.90 | OCF₃ | OCF₃ | NHCOM | OMe | 0 | |
| 2.91 | OCF₃ | OCF₃ | NHCOOEt | OMe | 1 | |
| 2.92 | OCF₃ | OCF₃ | NHCOOEt | H | 0 | |
| 2.93 | OCF₃ | OCF₃ | NHCOOEt | H | 1 | |
| 2.94 | CF₃ | CF₃ | NHCOOMe | OH | 1 | |
| 2.95 | CF₃ | CF₃ | NHCOO-i-prop. | OH | 0 | |
| 2.96 | OCF₃ | OCF₃ | NHCOO-i-prop. | OH | 0 | |
| 2.97 | CF₃ | CF₃ | 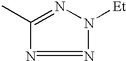 | OH | 0 | |
| 2.98 | CF₃ | CF₃ | 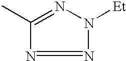 | OH | 1 | |
| 2.99 | CF₃ | CF₃ | NHCOO-i-prop. | OH | 1 | |
| 2.100 | OCF₃ | OCF₃ | NHCOO-i-prop. | OH | 1 | |
| 2.101 | CF₃ | CF₃ | NHCOO-i-but. | OH | 0 | |
| 2.102 | Cl | cl | NHCOO-i-but. | OH | 0 | |
| 2.103 | OCF₃ | CF₃ | 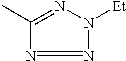 | OH | 0 | |
| 2.104 | OCF₃ | OCF₃ | 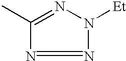 | OH | 0 | |
| 2.105 | OCF₃ | OCF₃ | 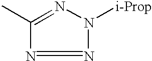 | OH | 0 | |
| 2.106 | OCF₃ | OCF₃ | 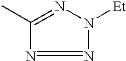 | OH | 1 | |
| 2.107 | OCF₃ | CF₃ | 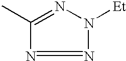 | OH | 1 | |
| 2.108 | Cl | cl | NHCOO-i-but. | OH | 1 | |
| 2.109 | CF₃ | CF₃ | NHCOO-i-but. | OH | 1 | |
| 2.110 | OCF₃ | OCF₃ | 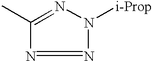 | OH | 1 | |
| 2.111 | CF₃ | CF₃ | 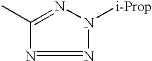 | OH | 0 | |
| 2.112 | CF₃ | CF₃ | 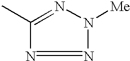 | OH | 0 | |
| 2.113 | OCF₃ | OCF₃ | NHCOO-i-but. | OH | 1 | |
| 2.114 | CF₃ | CF₃ | 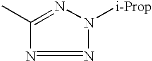 | OH | 1 | |
| 2.115 | CF₃ | CF₃ | 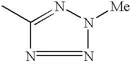 | OH | 1 | |
| 2.116 | OCF₃ | OCF₃ | 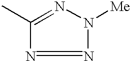 | OH | 0 | |
| 2.117 | OCF₃ | OCF₃ | 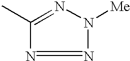 | OH | 1 | |
| 2.118 | CF₃ | CF₃ | 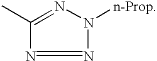 | OH | 0 | |
| 2.119 | CF₃ | CF₃ | 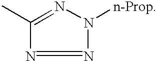 | OH | 1 | |
| 2.120 | OCF:3 | OCF₃ | 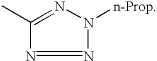 | OH | 0 | |
| 2.121 | OCF₃ | OCF₃ | 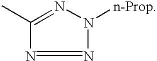 | OH | 1 | |
| 2.122 | CF₃ | F | NHCOO-i-prop. | OH | 0 | |
| 2.123 | OCF₃ | CF₃ | 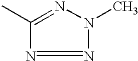 | OH | 0 | |
| 2.124 | OCF₃ | CF₃ | 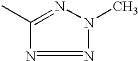 | OH | 1 | |
| 2.125 | CF₃ | CF₃ | —CH═NOCH₃ | OH | 0 | |
| 2.126 | CF₃ | CF₃ | —CH═NO₂H₅ | OH | 0 | |
| 2.127 | OCF₃ | OCF₃ | —CH═NO₂H₅ | OH | 0 | |
| 2.128 | OCF₃ | CF₃ | —CH═NO₂H₅ | OH | 0 | |
| 2.129 | OCF₃ | CF₃ | —CH═NOCH₃ | OH | 0 | |
| 2.130 | OCF₃ | CF₃ | —CH═NOC₂H₅ | OH | 1 | |
| 2.131 | OCF₃ | OCF₃ | —CH═NOC₂H₅ | OH | 1 | |
| 2.132 | CF₃ | CF₃ | 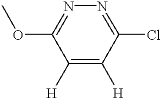 | OH | 0 | |
| 2.133 | CF₃ | CF₃ | 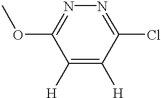 | OH | 1 | |
| 2.134 | t-But | t-But | NHCOO-i-prop. | OH | 0 | |
| 2.135 | t-But | t-But | NHCOO-i-prop. | OH | 1 | |
| 2.136 | CF₃ | CF₃ | 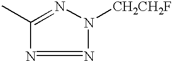 | OH | 0 | |
| 2.137 | CF₃ | CF₃ | 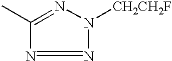 | OH | 0 | |
| 2.138 | OCF₃ | CF₃ | 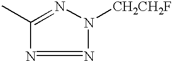 | OH | 0 | |
| 2.139 | OCF₃ | CF₃ | 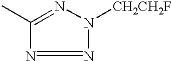 | OH | 1 | |
| 2.140 | OCF₃ | OCF₃ | 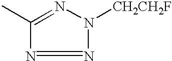 | OH | 0 | |
| 2.141 | OCF₃ | OCF₃ | 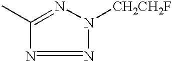 | OH | 1 | |
| 2.142 | CF₃ | CF₃ | 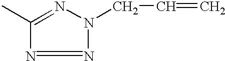 | OH | 0 | |
| 2.143 | CF₃ | CF₃ | 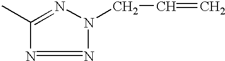 | OH | 1 | |
| 2.144 | CF₃ | CF₃ | 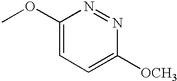 | OH | 0 | |
| 2.145 | CF₃ | CF₃ | 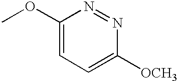 | OH | 1 | |
| 2.146 | CF₃ | CF₃ | 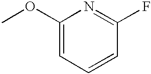 | OH | 0 | |
| 2.147 | CF₃ | CF₃ | 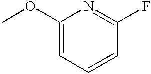 | OH | 1 | |
| 2.148 | OCF₃ | OCF₃ | 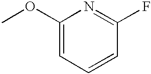 | OH | 0 | |
| 2.149 | OCF₃ | OCF₃ | 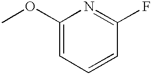 | OH | 1 | |
| 2.150 | CF₃ | CF₃ | phenyl | OH | 0 | |
| 2.151 | CF₃ | CF₃ | phenyl | OH | 1 | |
| 2.152 | F | F | OEt | OH | 0 | s.s. |
| 2.153 | F | F | O-n-prop. | OH | 0 | oil |
| 2.154 | F | F | —O-phenyl | OH | 0 | s.s. |
| 2.155 | F | F | OMe | OH | 0 | s.s. |
| 2.156 | F | F | OCF₃ | OH | 0 | s.s. |
| 2.157 | CF₃ | CF₃ | Ome | OH | 0 | oil |
| 2.158 | CF₃ | CF₃ | —O-phenyl | OH | 0 | oil |
| 2.159 | CF₃ | CF₃ | OCF₃ | OH | 0 | s.s. |
| 2.160 | OCF₃ | OCF₃ | —O-phenyl | OH | 0 | oil |
| 2.161 | OCF₃ | OCF₃ | OCF₃ | OH | 0 | oil |
| 2.162 | F | F | OEt | OH | 1 | s.s. |
| 2.163 | F | F | O-n-prop. | OH | 1 | s.s. |
| 2.164 | F | F | —O-phenyl | OH | 1 | s.s. |
| 2.165 | F | F | Ome | OH | 1 | s.s. |
| 2.166 | F | F | OCF₃ | OH | 1 | s.s. |
| 2.167 | CF₃ | CF₃ | Ome | OH | 1 | s.s. |
| 2.168 | CF₃ | CF₃ | —O-phenyl | OH | 1 | s.s. |
| 2.169 | CF₃ | CF₃ | OCF₃ | OH | 1 | s.s. |
| 2.170 | OCF₃ | OCF₃ | Ome | OH | 1 | s.s. |
| 2.171 | OCF₃ | OCF₃ | OEt | OH | 1 | s.s. |
| 2.172 | OCF₃ | OCF₃ | —O-phenyl | OH | 1 | s.s. |
| 2.173 | OCF₃ | OCF₃ | OCF₃ | OH | 1 | s.s. |

TABLE 3
Compounds of the formula (I.3)

| | |
|---|---|
| | (I.3) |
| 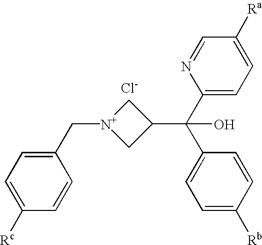 | |

In the compounds 3.1 to 3.35, the groups R, and Rc each have the same
meanings as in the corresponding compounds 1.1 to 1.35.

TABLE 4
Compounds of the formula (I.4)

| | |
|---|---|
| | (I.4) |
| 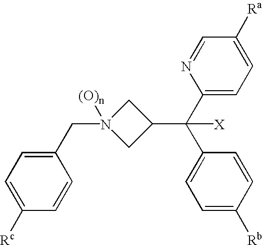 | |

In the compounds 4.1 to 4.173, the groups R^{a}, R^{b}, R^{c}, X and n each have
the same meanings as in the corresponding compounds 2.1 to 2.173.

TABLE 5
Compounds of the formula (I.5)

| | |
|---|---|
| | (I.5) |
| 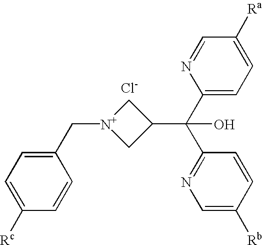 | |

In the compounds 5.1 to 5.35, the groups R^{a}, R^{b} and R^{c} each have the
same meanings as in the corresponding compounds 1.1 to 1.35.

TABLE 6
Compounds of the formula (I.6)

| | |
|---|---|
| | (1.6) |
| 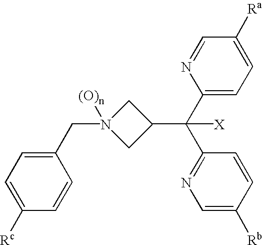 | |

In the compounds 6.1 to 6.173, the groups R^{a}, R^{b}, R^{c}, X and n each have
the same meanings as in the corresponding compounds 2.1 to 2.173.

### C. FORMULATION EXAMPLES

a) A dust is obtained by mixing 10 parts by weight of active compound and 90 parts by weight of talc as inert material and comminuting the mixture in a hammer mill.

b) A wettable powder which is readily dispersible in water is obtained by mixing 25 parts by weight of active substance, 65 parts by weight of kaolin-containing quartz as inert material, 10 parts by weight of potassium lignosulfonate and 1 part by weight of sodium oleoylmethyltaurinate as wetter and dispersant and grinding the mixture in a pinned-disk mill.

c) A dispersion concentrate which is readily dispersible in water is prepared by mixing 40 parts by weight of active substance with 7 parts by weight of a sulfosuccinic monoester, 2 parts by weight of a sodium lignosulfonate and 51 parts by weight of water and grinding the mixture in a ball mill to a fineness of below 5 microns.

d) An emulsifiable concentrate can be prepared from 15 parts by weight of active substance, 75 parts by weight of cyclohexane as solvent and 10 parts by weight of ethoxylated nonylphenol (10 EO) as emulsifier.

e) Granules can be prepared from 2 to 15 parts by weight of active substance and an inert granule carrier material such as attapulgite, pumice granules and/or quartz sand. It is expedient to use a suspension of the wettable powder of Example b) having a solids content of 30%, which is sprayed onto the surface of attapulgite granules and these are dried and mixed intimately. The wettable powder amounts to approx. 5% by weight and the inert carrier material to approx. 95% by weight of the finished granules.

### D. BIOLOGICAL EXAMPLES

In the examples A and B below, compounds are considered to be active when, at a concentration of 500 ppm or less, they show an activity against harmful organisms of 50% or more.

### Example A

A Petri dish whose bottom is covered with filter paper and which contains about 5 ml of nutrient medium is prepared. Pieces of filter paper with about 30 24-hour-old eggs of the American tobacco budworm (Heliothis virescens) are dipped for 5 seconds into an aqueous solution of the formulated preparation to be tested and are then placed into the Petri dish. A further 200 μl of the aqueous solution are distributed over the nutrient medium. The Petri dish is closed and then stored in a climatized chamber at about 25° C. After 6 days of storage, the effect of the preparation on the eggs and the larvae hatched therefrom (mortality) is determined. The compounds of the following examples were active: Nos. 2.158, 2.159, 2.5, 2.161, 2.49, 2.168, 2.17, 2.172, 2.171, 1.19, 2.106.

### Example B

A Petri dish whose bottom is covered with filter paper and which contains about 5 ml of nutrient medium is prepared. Five L2 larvae of the Egyptian cotton leafworm (Spodoptera littoralis) are counted into a small beaker. 200 μl of an aqueous solution of the formulated preparation to be tested are pipetted into the beaker. The treated larvae are then poured into the Petri dish, and a further 200 μl of the aqueous solution are distributed over the nutrient medium. The Petri dish is closed and then stored in a climatized chamber at about 25° C. After 6 days of storage, the effect of the preparation on the larvae (mortality) is determined. The compounds of the following examples were active: 2.158, 2.159, 2.5, 2.161, 2.49, 2.168, 2.17, 2.172, 2.171.

What is claimed is:

1. Azetidine derivatives of the formula (I) 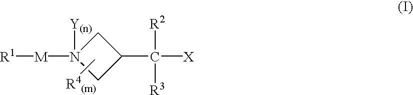
in which the symbols and indices are as defined below:
R¹ is aryl or heteroaryl, which are unsubstituted or mono- or polysubstituted by identical or different substituents;
R² and R³ are aryl or heteroaryl, which are unsubstituted or mono- or polysubstituted by identical or different substituents, it also being possible for the two groups to be bridged by a joint substituent;
M is unsubstituted or substituted (CH₂)ₗ, where l=1, 2 or 3, is CO or —HN—C(O);
X is H, OH, halogen, OR⁴ or CN;
Y is (O), H, OH, OR⁴, R⁴; in the case of the four lastmentioned groups—in which the nitrogen carries a positive charge—in combination with a corresponding anion;
R⁴ are identical or different (C₁-C₄)-alkyl, (C₁-C₄)-alkanoyl or (C₁-C₄)-haloalkyl groups;
m is 0, 1, 2, 3 or 4;
n is 0 or 1;
except for compounds in which
Y is CN,
R¹ is unsubstituted phenyl,
R² is unsubstituted phenyl,
R³ is unsubstituted phenyl or pyridyl and,
n is 0.

2. An azetidine derivative of the formula (I) as claimed in claim 1, where the symbols and indices are as defined below:
R¹, R², R³ independently of one another are carbocyclic aromatic radicals having 6 to 14 carbon atoms or aryl radicals having 6 to 14 ring members, in which at least one CH group is replaced by N and/or two adjacent CH groups are replaced by S, NH or O, where the radicals mentioned are unsubstituted or mono- or polysubstituted by identical or different radicals R⁵;
R⁴ are identical or different (C₁-C₄)-alkyl, (C₁-C₄)-alkanoyl or (C₁-C₄)-haloalkyl;
R⁵ are identical or different
c) halogen, CN, NO₂, SF₅;
d) straight-chain or branched alkyl groups having 1 to 12 carbon atoms, where one or more (CH₂) groups may be replaced by —O—, —S(O)—_{0,1,2}, —NH—, —NR⁶—, —CO—, —CS—, —CH═CH—, —C≡C—, unsubstituted or substituted aryldiyl, unsubstituted or substituted heterocyclyldiyl, unsubstituted or substituted (C₃-C₈)-cycloalkanediyl or unsubstituted or substituted (C₃-C₈)-cycloalkenediyl, with the proviso that chalcogen may not be adjacent, where two radicals R⁵ together with the atoms of the aromatic ring system may form a 3- to 8-membered ring system and where individual hydrogen atoms may be replaced by halogen, CN, NO₂ and/or SF₅;
with the proviso that the radical(s) R⁵ together do not comprise more than 3 ring systems having five or more members;
R⁶ is (C₁-C₄)-alkyl, unsubstituted or substituted phenyl or unsubstituted or substituted benzyl;
M is —CH₂—, —CH₂—CH₂—, —CH₂—CH₂—CH₂—, —CH(CH₃)—, —NH—CO—, —CO— or 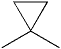
and
X is H, Cl, F, OH, CN.

3. An azetidine derivative of the formula (I) as claimed in claim 1, where the symbols and indices are as defined below:
R¹, R², R³ are identical or different phenyl, naphthyl, thiophene, furan, pyrrole, thiazole, oxazole, imidazole, isothiazole, isoxazole, pyrazole, 1,3,4-oxadiazole, 1,3,4-thiadiazole, 1,3,4-triazole, 1,2,4-oxadiazole, 1,2,4-thiadiazole, 1,2,4-triazole, 1,2,3-triazole, 1,2,3,4-tetrazole, benzo[b]thiophene, benzo[b]furan, indole, benzo[c]thiophene, benzo[c]furan, isoindole, benzoxazole, benzothiazole, benzimidazole, benzisoxazole, benzisothiazole, benzopyrazole, benzothiadiazole, benzotriazole, dibenzofuran, dibenzothiophene, carbazole, pyridine, pyrazine, pyrimidine, pyridazine, 1,3,5-triazine, 1,2,4-triazine, 1,2,4,5-triazine, quinoline, isoquinoline, quinoxaline, quinazoline, cinnoline, 1,8-naphthyridine, 1,5-naphthyridine, 1,6-naphthyridine, 1,7-naphthyridine, phthalazine, pyridopyrimidine, purine, pteridine or 4H-quinolizine groups which are unsubstituted or substituted by one or more radicals R⁵;
R⁴ are identical or different (C₁-C₄)-alkyl, (C₁-C₄)-alkanoyl or (C₁-C₄)-haloalkyl groups;
R⁵ are identical or different D-R⁷, or two radicals R⁵ together with the atoms to which they are attached form a three- to eight-membered saturated or unsaturated ring system which may also comprise further heteroatoms, preferably O, N, S, SO and/or SO₂, and which is unsubstituted or substituted by one or more radicals R⁷;
D is a direct bond or (C₁-C₆)-alkanediyl which is unsubstituted or substituted by one or more halogen atoms;
R⁷ are identical or different R⁸, R⁹, —C(W)R⁸, —C(═NOR⁸)R⁸, —C(═NR⁸)R⁸, —C(═NNR⁸₂)R⁸, —C(═W)OR⁸, —C(═W)NR⁸₂, —OC(═W)R⁸, —OC(═W)OR⁸, —NR⁸C(═W)R⁸, —N[C(═W)R⁸]₂, —NR⁸C(═W)OR⁸, —C(═W)NR⁸—NR⁸₂, —C(═W)NR⁸—NR⁸[C(═W)R⁸], —NR⁸—C(═W)NR⁸₂, —NR⁸—NR⁸C(═W)R⁸, —NR⁸—N[C(═W)R⁸]₂, —N[(C═W)R⁸]—NR⁸₂, —NR⁸—N[(C═W)WR⁸], —NR⁸[(C═W)NR⁸₂], —NR⁸(C═NR⁸)R⁸, —NR⁸(C═NR⁸)NR⁸₂, —O—NR⁸₂, —O—NR⁸(C═W)R⁸, —SO₂NR⁸₂, —NR⁸SO₂R⁸, —SO₂OR⁸, —OSO₂R⁸, —OR⁸, —NR⁸₂, —SR⁸, —SiR⁸₃, —PR⁸₂, —P(═W)R⁸₂, —SOR⁸, —SO₂R⁸, —PW₂R⁸₂, —PW₃R⁸₂ group;
W is O or S;
R⁸ are identical or different H, (C₁-C₆)-alkyl, (C₂-C₆)-alkenyl, (C₂-C₆)-alkynyl, (C₃-C₈)-cycloalkyl, (C₄-C₈)-cycloalkenyl, (C₃-C₈)-cycloalkyl-(C₁-C₄)-alkyl, (C₄-C₈)-cycloalkenyl-(C₁-C₄)-alkyl, (C₃-C₈)-cycloalkyl-(C₂-C₄)-alkenyl, (C₄-C₈)-cycloalkenyl-(C₂-C₄)-alkenyl, (C₁-C₆)-alkyl-(C₃-C₈)-cycloalkyl, (C₂-C₆)-alkenyl-(C₃-C₈)-cycloalkyl, (C₂-C₆)-alkynyl-(C₃-C₈)-cycloalkyl, (C₁-C₆)-alkyl-(C₄-C₈)-cycloalkenyl, (C₂-C₆)-alkenyl-(C₄-C₈)-cycloalkenyl, aryl or heterocyclyl groups, where the radicals mentioned are unsubstituted or substituted by one or more radicals R⁹ and two radicals R⁸ together may form a ring system;
R⁹ are identical or different halogen, cyano, nitro, hydroxyl, thio, amino, SF₅, formyl, (C₁-C₆)-alkanoyl, (C₁-C₆)-alkoxy, (C₃-C₆)-alkenyloxy, (C₃-C₆)-alkynyloxy, (C₁-C₆)-haloalkyloxy, (C₃-C₆)-haloalkenyloxy, (C₃-C₆)-haloalkynyloxy, (C₃-C₈)-cycloalkoxy, (C₄-C₈)-cycloalkenyloxy, (C₃-C₈)-halocycloalkoxy, (C₄-C₈)-halocycloalkenyloxy, (C₃-C₈)-cycloalkyl-(C₁-C₄)-alkoxy, (C₄-C₈)-cycloalkenyl-(C₁-C₄)-alkoxy, (C₃-C₈)-cycloalkyl-(C₂-C₄)-alkenyloxy, (C₄-C₈)-cycloalkenyl-(C₁-C₄)-alkenyloxy, (C₁-C₆)-alkyl-(C₃-C₈)-cycloalkoxy, (C₂-C₆)-alkenyl-(C₃-C₈)-cycloalkoxy, (C₂-C₆)-alkynyl-(C₃-C₈)-cycloalkoxy, (C₁-C₆)-alkyl-(C₄-C₈)-cycloalkenyloxy, (C₂-C₆)-alkenyl-(C₄-C₈)-cycloalkenyloxy, (C₁-C₄)-alkoxy-(C₁-C₆)-alkoxy, (C₁-C₄)-alkoxy-(C₃-C₆)-alkenyloxy, carbamoyl, (C₁-C₆)-mono- or -dialkylcarbamoyl, (C₁-C₆)-mono- or -dihaloalkylcarbamoyl, (C₃-C₈)-mono- or -dicycloalkylcarbamoyl, (C₁-C₆)-alkoxycarbonyl, (C₃-C₈)-cycloalkoxycarbonyl, (C₁-C₆)-alkanoyloxy, (C₃-C₈)-cycloalkanoyloxy, (C₁-C₆)-haloalkoxycarbonyl, (C₁-C₆)-haloalkanoyloxy, (C₁-C₆)-alkaneamido, (C₁-C₆)-haloalkaneamido, C(O)NH—(C₁-C₆)-alkyl, C(O)NH—(C₁-C₆)-haloalkyl, C(O)N-[(C₁-C₆)-alkyl]₂, C(O)N-[(C₁-C₆)-haloalkyl]₂, (C₂-C₆)-alkeneamido, (C₃-C₈)-cycloalkaneamido, (C₃-C₈)-cycloalkyl-(C₁-C₄)-alkaneamido, (C₁-C₆)-alkylthio, (C₃-C₆)-alkenylthio, (C₃-C₆)-alkynylthio, (C₁-C₆)-haloalkylthio, (C₃-C₆)-haloalkenylthio, (C₃-C₆)-haloalkynylthio, (C₃-C₈)-cycloalkylthio,
(C₄-C₈)-cycloalkenylthio, (C₃-C₈)-halocycloalkylthio, (C₄-C₈)-halocycloalkenylthio, (C₃-C₈)-cycloalkyl-(C₁-C₄)-alkylthio, (C₄-C₈)-cycloalkenyl-(C₁-C₄)-alkylthio, (C₃-C₈)-cycloalkyl-(C₃-C₄)-alkenylthio, (C₄-C₈)-cycloalkenyl-(C₃-C₄)-alkenylthio, (C₁-C₆)-alkyl-(C₃-C₈)-cycloalkylthio, (C₂-C₆)-alkenyl-(C₃-C₈)-cycloalkylthio, (C₂-C₆)-alkynyl-(C₃-C₈)-cycloalkylthio, (C₁-C₆)-alkyl-(C₄-C₈)-cycloalkenylthio, (C₂-C₆)-alkenyl-(C₄-C₈)-cycloalkenylthio, (C₁-C₆)-alkylsulfinyl, (C₃-C₆)-alkenylsulfinyl, (C₃-C₆)-alkynylsulfinyl, (C₁-C₆)-haloalkylsulfinyl, (C₃-C₆)-haloalkenylsulfinyl, (C₃-C₆)-haloalkynylsulfinyl, (C₃-C₈)-cycloalkylsulfinyl, (C₄-C₈)-cycloalkenylsulfinyl, (C₃-C₈)-halocycloalkylsulfinyl, (C₄-C₈)-halocycloalkenylsulfinyl, (C₃-C₈)-cycloalkyl-(C₁-C₄)-alkylsulfinyl, (C₄-C₈)-cycloalkenyl-(C₁-C₄)-alkylsulfinyl, (C₃-C₈)-cycloalkyl-(C₃-C₄)-alkenylsulfinyl, (C₄-C₈)-cycloalkenyl-(C₃-C₄)-alkenylsulfinyl, (C₁-C₆)-alkyl-(C₃-C₈)-cycloalkylsulfinyl, (C₂-C₆)-alkenyl-(C₃-C₈)-cycloalkylsulfinyl, (C₂-C₆)-alkynyl-(C₃-C₈)-cycloalkylsulfinyl, (C₁-C₆)-alkyl-(C₄-C₈)-cycloalkenylsulfinyl, (C₂-C₆)-alkenyl-(C₄-C₈)-cycloalkenylsulfinyl, (C₁-C₆)-alkylsulfonyl, (C₃-C₆)-alkenylsulfonyl, (C₃-C₆)-alkynylsulfonyl, (C₁-C₆)-haloalkylsulfonyl, (C₃-C₆)-haloalkenylsulfonyl, (C₃-C₆)-haloalkynylsulfonyl, (C₃-C₈)-cycloalkylsulfonyl, (C₄-C₈)-cycloalkenylsulfonyl, (C₃-C₈)-halocycloalkylsulfonyl, (C₄-C₈)-halocycloalkenylsulfonyl, (C₃-C₈)-cycloalkyl-(C₁-C₄)-alkylsulfonyl, (C₄-C₈)-cycloalkenyl-(C₁-C₄)-alkylsulfonyl, (C₃-C₈)-cycloalkyl-(C₃-C₄)-alkenylsulfonyl, (C₄-C₈)-cycloalkenyl-(C₃-C₄)-alkenylsulfonyl, (C₁-C₆)-alkyl-(C₃-C₈)-cycloalkylsulfonyl, —(C₂-C₆)-alkenyl-(C₃-C₈)-cycloalkylsulfonyl, (C₂-C₆)-alkynyl-(C₃-C₈)-cycloalkylsulfonyl, (C₁-C₆)-alkyl-(C₄-C₈)-cycloalkenylsulfonyl, (C₂-C₆)-alkenyl-(C₄-C₈)-cycloalkenylsulfonyl, (C₁-C₆)-dialkylamino, (C₁-C₆)-alkylamino, (C₃-C₆)-alkenylamino, (C₃-C₆)-alkynylamino, (C₁-C₆)-haloalkylamino, (C₃-C₆)-haloalkenylamino, (C₃-C₆)-haloalkynylamino, (C₃-C₈)-cycloalkylamino, (C₄-C₈)-cycloalkenylamino, (C₃-C₈)-halocycloalkylamino, (C₄-C₈)-halocycloalkenylamino, (C₃-C₈)-cycloalkyl-(C₁-C₄)-alkylamino, (C₄-C₈)-cycloalkenyl-(C₁-C₄)-alkylamino, (C₃-C₈)-cycloalkyl-(C₃-C₄)-alkenylamino, (C₄-C₈)-cycloalkenyl-(C₃-C₄)-alkenylamino, (C₁-C₆)-alkyl-(C₃-C₈)-cycloalkylamino, (C₂-C₆)-alkenyl-(C₃-C₈)-cycloalkylamino, (C₂-C₆)-alkynyl-(C₃-C₈)-cycloalkylamino, (C₁-C₆)-alkyl-(C₄-C₈)-cycloalkenylamino, (C₂-C₆)-alkenyl-(C₄-C₈)-cycloalkenylamino, (C₁-C₆)-trialkylsilyl, aryl, aryloxy, arylthio, arylamino, aryl-(C₁-C₄)-alkoxy, aryl-(C₃-C₄)-alkenyloxy, aryl-(C₁-C₄)-alkylthio, aryl-(C₂-C₄)-alkenylthio, aryl-(C₁-C₄)-alkylamino, aryl-(C₃-C₄)-alkenylamino, aryl-(C₁-C₆)-dialkylsilyl, diaryl-(C₁-C₆)-alkylsilyl, triarylsilyl or 5- or 6-membered heterocyclyl groups, where the cyclic radicals are unsubstituted or substituted by one or more radicals selected from the group consisting of halogen, cyano, nitro, amino, hydroxy, thio, (C₁-C₄)-alkyl, (C₁-C₄)-haloalkyl, (C₃-C₈)-cycloalkyl, (C₁-C₄)-alkoxy, (C₁-C₄)-haloalkoxy, (C₁-C₄)-alkylthio, (C₁-C₄)-haloalkylthio, (C₁-C₄)-alkylamino, (C₁-C₄)-haloalkylamino, formyl and (C₂-C₄)-alkanoyl;
M is —CH₂—, —CH₂—CH₂— or —CH₂—CH₂—CH₂—;
X is F, OH or H and
Y is O, OH or H.

4. An azetidine derivative of the formula (I) as claimed in claim 1, where the symbols and indices are as defined below:
R¹ is phenyl, pyridyl, pyrimidyl, thienyl or furanyl, unsubstituted or mono- or polysubstituted by halogen, (C₁-C₄)-alkoxy, (C₁-C₄)-haloalkoxy, (C₃-C₆)-cyclopropyl-(C₁-C₄)-alkoxy, (C₃-C₆)-alkenyloxy, (C₁-C₄)-alkoxycarbonyl, —NHCO₂—(C₁-C₄)-alkyl, —NHCO₂—(C₁-C₄)-haloalkyl, —N═C((C₁-C₄)-alkyl)-COO(C₁-C₄)-alkyl, —N((C₁-C₄)-alkyl)-COO—(C₁-C₄)-haloalkyl, OC(O)NH—(C₁-C₄)-alkyl, —O—C(O)—N((C₁-C₄)-alkyl)₂, CH═NO—(C₁-C₄)-alkyl, —CH═N—(C₁-C₄)-alkyl, CH═N-phenyl, CH═N-heterocyclyl, aryloxy, 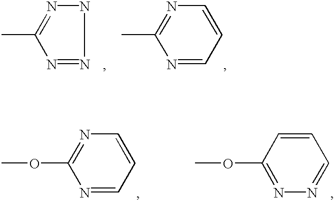
where the 5 lastmentioned groups are unsubstituted or mono- or—if possible—polysubstituted by identical or different radicals selected from the group consisting of halogen, (C₁-C₄)-alkyl, (C₁-C₄)-haloalkyl, (C₁-C₄)-alkoxy and (C₁-C₄)-haloalkoxy, or two radicals together are —O—(CH₂)_{1,2}—O—;
R² R³ are identical or different phenyl, pyridyl, thienyl or furanyl groups which are unsubstituted or mono- or polysubstituted by halogen, SF₅, (C₁-C₄)-haloalkyl or (C₁-C₄)-haloalkoxy;
R⁴ are identical or different (C₁-C₄)-alkyl, (C₁-C₄)-alkanoyl or (C₁-C₄)-haloalkyl groups;
M is —CH₂—, —CH₂—CH₂— or —CH₂—CH₂—CH₂—;
X is F, OH or H;
Y is O, OH or H.

5. An azetidine derivative of the formula (I) as claimed in claim 1, where the symbols and indices are as defined below:
R¹ is phenyl which is unsubstituted or mono- or polysubstituted by identical or different substituents from the group consisting of OEt, OⁿPr, OⁿBu, OⁱBu, O^{t}Bu, OCF₃, OCH₂CF₃, halogen, 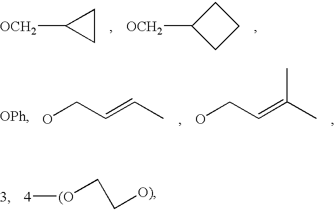
CO₂Et, NHCO₂Me, NHCO₂Et, NHCO₂ⁱPr, NHCO₂ⁿBu, NHCO₂CH₂CF₃, NⁱPrCO₂Me, OCO₂NMe₂, OCO₂NHMe, OCO₂NHEt, CH═NOEt, CH═NOⁿPr, CH═NMe, CH═NEt or CH═NⁱPr, 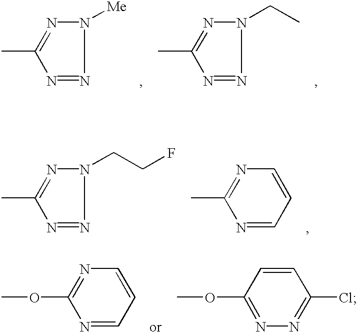
R² is phenyl or pyridyl, unsubstituted or mono- or polysubstituted by identical or different substituents selected from the group consisting of CF₃, OCF₃, F, Cl, Br, I, SF₅, OCHF₂, OCH₂₂CF₃;
M is CH₂;
X is OH;
Y is O, OH or H;
R⁴ is Me;
m is 0, 1 or 2 and
n is 0 or 1.

6. A process for preparing an azetidine derivative of the formula (I) as claimed in claim 1, wherein
b) an ester (VIII) 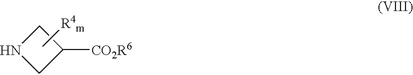
where the symbols and indices are as defined in formula (I) is converted by acylation, alkylation or reductive amination into intermediate (II) 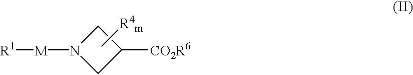
where the symbols and indices are as defined in formula (I); and
b1) if, in the formula (I), R² equals R³,
intermediate (II) is, in a reaction with Li or Mg and a compound R²G¹, where G¹ is Cl, Br or I and R² as defined in formula (I), converted into an azetidine derivative of the formula (V) 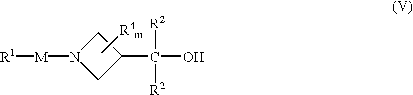
in which the symbols and indices are as defined in formula (I); or
b2) if, in formula (I), R² is not equal to R³, intermediate (II) is, by hydrolysis, activation of the acid and subsequent reaction with HNMe(OMe), converted into amide (IX) 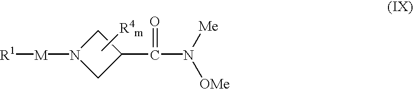
where the symbols and indices are as defined in the formula (I)
and amide (IX) is, by subsequent reactions with R²G¹ and Li or Mg or R³G¹ and Li or Mg, where the radicals G¹ are identical or different and are Cl, Br or I and R² and R³ are as defined in the formula (I), converted further into azetidine derivatives of the formula (Va) 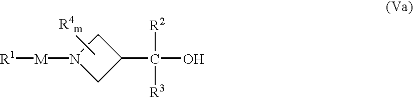
and
c) the azetidine derivatives (V) or (Va) are, if appropriate, converted further, according to known methods, into azetidine derivatives of the formula (I) where X≠OH and/or n≠0.

7. A pesticide, which comprises at least one compound as claimed in claim 1 and at least one formulation auxiliary.

8. An insecticidal, acaricidal or nematicidal composition as claimed in claim 7, which comprises an effective amount of at least one compound as claimed in claim 1 together with additives or auxiliaries customary for this application.

9. A pesticide, which comprises an insecticidally, acaricidally or nematocidally effective amount of at least one compound as claimed in claim 1 and at least one further active compound together with auxiliaries and additives customary for this application.

10. A composition for use in wood protection or as a preservative in sealants, in paints, in cooling lubricants for metalworking or in drillings and cutting oils, comprising an effective amount of at least one compound as claimed in claim 1 together with auxiliaries and additives customary for these applications.

11. A compound as claimed in claim 1 for preparing a veterinary medicament.

12. A composition as claimed in claim 7 for preparing a veterinary medicament.

13. A process for preparing a composition as claimed in claim 7, which comprises mixing the active compound and the further additives and bringing them into a suitable use form.

14. A method for controlling harmful insects, Acarina, molluscs and nematodes which comprises applying an effective amount of a compound as claimed in claim 1 to these or to the plants, areas or substrates infested with them.

15. A method for controlling harmful insects, Acarina, molluscs and nematodes which comprises applying an effective amount of a composition as claimed in claim 7 to these or to the plants, areas or substrates infested with them.

16. Seed, treated or coated with an effective amount of a compound as claimed in claim 1.

17. Seed, treated or coated with an effective amount of a composition as claimed in claim 7.
